(19) United States
(12) Patent Application Publication
(10) Pub. No.: US 2025/0161920 A1
(43) Pub. Date: May 22, 2025

(54) OXO DICOPPER ANCHORED ON CARBON NITRIDE FOR SELECTIVE OXIDATION OF METHANE

(71) Applicant: The Johns Hopkins University, Baltimore, MD (US)

(72) Inventors: Chao WANG, Ellicott City, MD (US); Pengfei XIE, Baltimore, MD (US)

(21) Appl. No.: 18/839,472

(22) PCT Filed: Feb. 27, 2023
(86) PCT No.: PCT/US2023/063330

§ 371 Date: Aug. 19, 2024

Related U.S. Application Data
(60) Provisional application No. 63/313,897, filed on Feb. 25, 2022.

(51) Int. Cl. B01J 23/72; B01J 27/24; B01J 35/39; B01J 37/04; B01J 37/08; C07C 29/50

(52) U.S. Cl. CPC B01J 23/72; B01J 27/24; B01J 35/39; B01J 37/04; B01J 37/086; C07C 29/50

(57) ABSTRACT

Disclosed are dimeric copper centers supported on graphitic carbon nitride (denoted herein as Cu₂@C₃N₄) and their use as advanced catalysts for partial oxidation of CH₄.

### FEDERALLY SPONSORED RESEARCH OR DEVELOPMENT

This invention was made with government support under grant DE-AR0000952 awarded by the Department of Energy. The government has certain rights in the invention.

### BACKGROUND

Selective conversion of methane to liquid hydrocarbons represents a promising approach toward efficient utilization of natural gas. Nisbet and Bousquet, 2014. The present industrial route for such conversions relies on a two-step process by first reforming methane to generate synthesis gas (CO and H₂) at elevated temperatures (greater than 500° C.), and then reacting CO with H₂ to form methanol or other liquid products. Choudhary and Choudhary, 2008; Tang et al., 2014. This process, however, is energy-intensive and economically nonviable for distributed sources such as flare gas. Buzcu-Guven and Harriss, 2012. More robust technologies toward direct conversion of methane into condensed energy carriers are needed to facilitate transportation and storage. Julian-Duran et al., 2014.

### SUMMARY

In some aspects, the presently disclosed subject matter provides a catalyst composite comprising a copper dimer having two copper atoms bridged by an oxygen atom, wherein each copper atom is coordinated to two nitrogen atoms of a carbon nitride substrate.

In certain aspects, the carbon nitride substrate comprises one or more of graphitic carbon nitride, α-carbon nitride, β-carbon nitride, cubic carbon nitride, pseudocubic carbon nitride, and combinations thereof. In particular aspects, the carbon nitride substrate comprises graphitic carbon nitride. In more particular aspects, the graphitic carbon nitride has a form selected from a film, a sphere, a nanotube, a nanorod, a nanosized powder, and combinations thereof.

In certain aspects, the catalyst composite comprises a loading of Cu of about 0.35 wt % of the total weight of the composite.

In particular aspects, the catalyst composite has an N 1s X-ray photoelectron spectroscopy (XPS) spectrum exhibiting a binding energy ranging from about 397 eV to about 408 eV comprising four peaks centered at about 398.6 eV, about 399.4 eV, about 401.0 eV, and about 404.5 eV. In particular aspects, the catalyst composite has a Cu 2p X-ray photoelectron spectroscopy (XPS) spectrum exhibiting peaks at 932.5 eV and 952.3 eV. In particular aspects, the catalyst composite has an Absorption Near Edge Spectroscopy (XANES) Cu K-edge spectrum exhibiting a pre-edge transition at about 8,984 eV.

In certain aspects, the two copper atoms have an oxidation state between +1 and +2. In particular aspects, the two copper atoms have an oxidation state of about +1.63 and about +1.72.

In certain aspects, the catalyst composite has an average distance between the two copper atoms is between about 0.18 nm to about 0.38 nm. In particular aspects, the average distance between the two copper atoms is about 0.28 nm+0.02 nm.

In other aspects, the presently disclosed subject matter provides a method for preparing the catalyst composite of claim 1, the method comprising:
(a) providing a copper-dimer organometallic precursor;
(b) mixing the copper dimer organometallic precursor with a carbon nitride substrate to form a mixture; and
(c) heating the mixture to form a catalyst composite comprising a copper dimer having two copper atoms bridged by an oxygen atom, wherein each copper atom is coordinated to two nitrogen atoms of a carbon nitride substrate.

In certain aspects, the copper-dimer organometallic precursor comprises an (oxalato) (bipyridine) copper (II) complex (Cu₂(bpy)₂(μ-ox)]Cl₂). In particular aspects, the Cu₂(bpy)₂(μ-ox)]Cl₂ is prepared by reacting copper chloride (CuCl₂) with 2,2′,-bipyridine and oxalic acid.

In certain aspects, the carbon nitride substrate comprises one or more of graphitic carbon nitride, α-carbon nitride, β-carbon nitride, cubic carbon nitride, pseudocubic carbon nitride, and combinations thereof. In particular aspects, the carbon nitride substrate comprises graphitic carbon nitride. In more particular aspects, the graphitic carbon nitride is prepared by calcination of urea.

In certain aspects, the method for preparing the catalyst composite comprises heating the mixture from about 50° C. to about 250° C. for about 10 hours at a rate of about 2° C. min⁻¹.

In other aspects, the presently disclosed subject matter provides a method for oxidizing methane, the method comprising contacting methane with the presently disclosed catalyst composite in the presence of an oxidizing agent.

In certain aspects, the oxidizing agent comprises H₂O₂. In particular aspects, the method comprises thermocatalytic oxidation of CH₄. In more particular aspects, the methane, catalyst composite, and oxidizing agent are maintained at a predetermined pressure and temperature for a period of time.

In certain aspects, the oxidizing agent comprises O₂. In particular aspects, the method comprises photocatalytic oxidation of CH₄. In more particular aspects, the methane, catalyst composite, and oxidizing agent are irradiated with visible light at a predetermined pressure and temperature for a period of time.

In certain aspects, the method of oxidizing CH₄ further comprises forming one or more methyl oxygenates. In particular aspects, the one or more methyl oxygenates are selected from methanol (CH₃OH) and methyl hydroperoxide (CH₃OOH). In more particular aspects, the method further comprises reducing the methyl hydroperoxide (CH₃OOH) to methanol (CH₃OH).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1a, FIG. 1b, FIG. 1c, FIG. 1d, FIG. 1e, FIG. 1f, FIG. 1g, and FIG. 1h illustrate the synthesis of the presently disclosed Cu₂@C₃N₄ catalysts. (FIG. 1a) Scheme of a representative synthetic route. (FIG. 1b, FIG. 1c) Characterization of the Cu-dimer precursor, [Cu₂(bpy)₂(μ-ox)]Cl₂ complex using FTIR (FIG. 1b) and UV-vis DRS. (FIG. 1d) Comparison of FTIR spectra for Cu₂@C₃N₄ and g-C₃N₄. (FIG. 1e, FIG. 1f, FIG. 1g) Representative HAADF-STEM images of Cu₂@C₃N₄, with the insets showing line-scanning intensity profiles of Cu dimers. (FIG. 1h) Statistical distribution of the Cu—Cu distance in the Cu dimers derived from the STEM images:

FIG. 2a, FIG. 2b, FIG. 2c, FIG. 2d, FIG. 2e, and FIG. 2f show the characterization of the presently disclosed Cu₂@C₃N₄ catalysts. (FIG. 2a) XPS spectrum at the N 1s edge and the corresponding deconvolution. (FIG. 2b) XANES spectra and (FIG. 2c) k²-weighted EXAFS spectra at the Cu K edge, with Cu foil, Cu₂O, CuO and Cu-TPP (one Cu coordinated with for N atoms) as the reference. (FIG. 2d) Fitting of the EXAFS spectrum with consideration of both monomeric and dimeric Cu sites. (FIG. 2e) The simulated structure model of dicopper-oxo center. (FIG. 2f) Geometric parameters of the dicopper-oxo center determined for Cu₂@C₃N₄;

FIG. 3a, FIG. 3b, FIG. 3c, FIG. 3d, and FIG. 3e show thermocatalytic oxidation of CH₄ with H₂O₂. (FIG. 3a) Yields and productivity of methyl oxygenates at different reaction temperatures. (FIG. 3b) Comparisons of product yields and productivity over different catalysts. (FIG. 3c) Correlation between productivity of methyl oxygenates and gain factor for different catalysts. (FIG. 3d) Simulated pathways for the reaction between CH₄ with H₂O₂ on the Cu₂@C₃N₄ catalysts, with the middle inset illustrating the electron distribution of the CH₄ molecule being activated on the bridging oxygen site. Energy barriers also are given for the associated molecular transformations. (FIG. 3e) The DFT calculated free energy diagram for the Cu₂@C₃N₄-catalyzed partial oxidation of CH₄ with H₂O₂. Three stages consisting of H₂O₂ activation, CH₄ activation and methyl oxygenates formation are distinguished with different colors. The error bars presented in (FIG. 3a, FIG. 3b, and FIG. 3c) indicate the statistical distribution derived from three independent measurements;

FIG. 4a, FIG. 4b, FIG. 4c, FIG. 4d, FIG. 4e, and FIG. 4f show the photocatalytic oxidation of CH₄ with O₂. (FIG. 4a) Yields and productivity of methyl oxygenates as a function of reaction time at 0.1 MPa CH₄ and 0.1 MPa O₂. (FIG. 4b) CH₄ conversions and productivity of methyl oxygenates at different CH₄ and O₂ partial pressures. (FIG. 4c) EPR spectra recorded for the various control experiments using DMPO as the radical trapping agent. (FIG. 4d, FIG. 4e) In situ irradiation XPS spectra collected at the O 1s (FIG. 4d) and N 1s (e) edges. (FIG. 4f) Schematic illustration of the photocatalytic oxidation of CH₄ with O₂ catalyzed by Cu₂@C₃N₄. The values “−1.45 and 1.31 eV” label the estimated position of dicopper-oxo states in the band structure of g-C₃N₄, as determined by performing Tauc plot analysis on the UV-vis DRS and UPS spectra of Cu₂@C₃N₄. The error bars shown in (FIG. 4a, FIG. 4b) indicate the statistical distribution derived from three independent measurements;

FIG. 5a and FIG. 5b show the thermogravimetric analysis (TGA) profiles showing the weight loss and the corresponding first derivative values of the dimeric copper complex (FIG. 5a) and the pristine Cu₂@C₃N₄ with ligands (FIG. 5b) when burned in air;

FIG. 6 shows the XRD patterns of Cu₂@C₃N₄ and g-C₃N₄;

FIG. 7 shows an XPS spectrum of Cu 2p edge for Cu₂@C₃N₄;

FIG. 8 shows the Cu K-edge XANES spectra of Cu₂@C₃N₄, Cu foil, Cu₂₀, CuO and copper tetraphenylporphyrin (Cu-TPP). Inset: the molecular structure of Cu-TPP which involves one Cu coordinated to four N via Cu—N bonding;

FIG. 9a and FIG. 9b show alternative Cu-dimer configurations simulated by using DFT and comparison of the corresponding EXAFS fitting to experimental spectra;

FIG. 10 is the k-space fitting analysis of EXAFS spectrum for Cu₂@C₃N₄ with consideration of both monomeric and dimeric Cu sites. This fitting analysis corresponds to FIG. 2d. The corresponding fitting parameters are summarized in Table 1;

FIG. 11a and FIG. 11b show the Bader charge analysis for the oxidation state of Cu in Cu₂@C₃N₄. The error bars in FIG. 11b indicate the statistical distribution of the computed Cu charges. Fitting the Bader charge of Cu derived from DFT calculations into a calibration curve established based on the references of metallic Cu, Cu₂O and CuO gives an oxidation state of +1.67, which is consistent with experimental results based on XPS and XANES;

FIG. 12 is a representative NMR spectrum collected for the methane oxidation products using Cu₂@C₃N₄ catalysts and H₂O₂ as the oxidizer;

FIG. 13 is a calibration curve for the analysis of CH₃OOH using NMR. The error bars indicate the statistical distribution derived from three independent measurements;

FIG. 14 is a calibration curve for the analysis of CH₃OH using NMR. The error bars indicate the statistical distribution derived from three independent measurements;

FIG. 15 is a calibration curve for the GC analysis of CO₂. The error bars indicate the statistical distribution derived from three independent measurements;

FIG. 16 shows the room temperature reduction of CH₃OOH with NaBH₄ to form CH₃OH;

FIG. 17a and FIG. 17b show the CH₄ conversion and product selectivity of the thermocatalytic oxidation of methane using H₂O₂ and Cu₂@C₃N₄. (FIG. 13a) Dependence on reaction temperature and (FIG. 13b) time (at 50° C.). The results indicate that evaluated temperature and prolongated reaction time lead to the overoxidation of CH₄ to CO₂, which is the thermodynamically most stable product. The error bars indicate the statistical distribution derived from three independent measurements;

FIG. 18 show the cycling test of Cu₂@C₃N₄ for thermocatalytic oxidation of CH₄ with H₂O₂. The error bars indicate the statistical distribution derived from three independent measurements;

FIG. 19a, FIG. 19b, FIG. 19c, and FIG. 19d show the characterization of the spent Cu₂@C₃N₄ catalyst after the 6-h durability test. (FIG. 19a) Representative HAADF-STEM images with Cu dimers highlighted using red circles. (FIG. 19b) k²-weighted EXAFS spectra at the Cu K edge, with Cu foil, Cu₂O, CuO and Cu-TPP (one Cu coordinated with for N atoms) being used as the references. (FIG. 19c, FIG. 19d) Fitting of the EXAFS spectrum with consideration of both monomeric and dimeric Cu sites;

FIG. 20 is an NMR spectrum collected for the control experiment using bare g-C₃N₄. No oxidation product was detected;

FIG. 21a, FIG. 21b, FIG. 21c, FIG. 21d, and FIG. 21e. show (FIG. 21a, FIG. 21b) XANES and (FIG. 21c) EXAFS spectra collected at the Cu K edge for Cu₁@C₃N₄. Cu foil, Cu₂₀, CuO and Cu-TPP (1 Cu coordinated with 4N atoms) also were shown as references. (FIG. 21d) The optimized structure of Cu₁@C₃N₄ based on DFT calculations. (FIG. 21e) Fitting parameters for the EXAFS spectrum of Cu₁@C₃N₄;

FIG. 22 is the calibration curve of H₂O₂ quantified by the titration of Ce(SO₄)₂. The error bars indicate the statistical distribution derived from three independent experimental measurements;

FIG. 23 shows the comparisons of H₂O₂ consumption and gain factors (denoted as mol of CH₃OH and CH₃OOH divided by total mol of H₂O₂ consumed) over different catalysts. The error bars indicate the statistical distribution derived from three independent experimental measurements;

FIG. 24 is the free energy diagram for the second reaction pathway of methane partial oxidation to CH₃OH on fresh Cu₂@C₃N₄ catalysts;

FIG. 25a and FIG. 25b are the EPR spectra of the radicals ·OOH in methanol (FIG. 21a) and ·OH in H₂O (FIG. 21b) at different conditions with DMPO as the radical trapping agent, showing that the presence of Cu₂@C₃N₄ enhances the cleavage of H₂O₂ to ·OOH and ·OH;

FIG. 26 is the comparison of the barriers for H₂O₂ cleavage to ·OOH and ·OH catalyzed by different catalysts;

FIG. 27 shows the in situ EPR characterization of thermal catalytic CH₄ selective oxidation by H₂O₂ on Cu₂@C₃N₄ with DMPO as radical trapping agent;

FIG. 28a, FIG. 28b, and FIG. 28c show (FIG. 28a, FIG. 28b) Experimental set-up of photocatalytic CH₄ oxidation by O₂. (FIG. 28c) Schematic illustration of the process for photocatalytic CH₄ oxidation by O₂:

FIG. 29 shows the product selectivity of photocatalytic CH₄ oxidation with O₂ over Cu₂@C₃N₄ as a function of reaction time. The error bars indicate the statistical distribution derived from three independent experimental measurements;

FIG. 30 shows the product selectivity of photocatalytic CH₄ oxidation by O₂ over Cu₂@C₃N₄ as function of CH₄/O₂ ratios. The error bars indicate the statistical distribution derived from three independent experimental measurements;

FIG. 31 shows the in situ irradiation XPS characterizations of Cu 2p edge on Cu₂@C₃N₄ under dark and visual light;

FIG. 32a and FIG. 32b show the (FIG. 32a) UV-vis DRS results of C₃N₄ and hydrated Cu₂@C₃N₄ and (FIG. 32b) corresponding Tauc plots of C₃N₄ and hydrated Cu₂@C₃N₄; and

FIG. 33 shows the UPS results of C₃N₄ and hydrated Cu₂@C₃N₄.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Oxo Dicopper Anchored on Carbon Nitride for Selective Oxidation of Methane

In some embodiments, the presently disclosed subject matter provides dimeric copper centers supported on graphitic carbon nitride (denoted herein as Cu₂@C₃N₄) as advanced catalysts for the partial oxidation of CH₄. The copper-dimer catalysts demonstrate high selectivity for partial oxidation of methane under both thermo- and photo-catalytic reaction conditions, with hydrogen peroxide (H₂O₂) and oxygen (O₂) being used as the oxidizer, respectively. In particular, the photocatalytic oxidation of CH₄ with O₂ achieves greater than 10% conversion and greater than 98% selectivity toward methyl oxygenates and a mass-specific activity of 1399.3 mmol gCu⁻¹ h⁻¹.

A comprehensive comparison to the literature results under similar reaction conditions indicate that the presently disclosed results represent the highest activity for partial oxidation of methane, with improvement factors of at least greater than 10. The presently disclosed copper-dimer catalysts were first evaluated for thermal oxidation of methane using H₂O₂ as the oxidizer and then further applied for photocatalytic oxidation of methane with O₂.

Accordingly, in some embodiments, the presently disclosed subject matter provides a catalyst composite comprising a copper dimer having two copper atoms bridged by an oxygen atom, wherein each copper atom is coordinated to two nitrogen atoms of a carbon nitride substrate.

In certain embodiments, the carbon nitride substrate comprises one or more of graphitic carbon nitride, α-carbon nitride, β-carbon nitride, cubic carbon nitride, pseudocubic carbon nitride, and combinations thereof. In particular embodiments, the carbon nitride substrate comprises graphitic carbon nitride. In more particular embodiments, the graphitic carbon nitride has a form selected from a film, a sphere, a nanotube, a nanorod, a nanosized powder, and combinations thereof.

In certain embodiments, the catalyst composite comprises a loading of Cu of about 0.35 wt % of the total weight of the composite, including about 0.25 wt % to about 0.5 wt %, including about 25, 26, 27, 28, 29, 30, 31, 32, 33, 34, 35, 36, 37, 38, 39, 40, 41, 42, 43, 44, 45, 46, 47, 48, 49, and 50 wt %.

In particular embodiments, the catalyst composite has an N 1s X-ray photoelectron spectroscopy (XPS) spectrum exhibiting a binding energy ranging from about 397 eV to about 408 eV comprising four peaks centered at about 398.6 eV, about 399.4 eV, about 401.0 eV, and about 404.5 eV. In particular embodiments, the catalyst composite has a Cu 2p X-ray photoelectron spectroscopy (XPS) spectrum exhibiting peaks at 932.5 eV and 952.3 eV. In particular embodiments, the catalyst composite has an Absorption Near Edge Spectroscopy (XANES) Cu K-edge spectrum exhibiting a pre-edge transition at about 8,984 eV.

In certain embodiments, the two copper atoms have an oxidation state between +1 and +2, including about +1, +1.1, +1.2, +1.3, +1.4, +1.5, +1.6, +1.7, +1.8, +1.9, and +2. In particular embodiments, the two copper atoms have an oxidation state of about +1.63 and about +1.72.

In certain embodiments, the catalyst composite has an average distance between the two copper atoms is between about 0.18 nm to about 0.38 nm, including about 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, and 0.38 nm. In particular embodiments, the average distance between the two copper atoms is about 0.28 nm±0.02 nm.

In other embodiments, the presently disclosed subject matter provides a method for preparing the catalyst composite of claim 1, the method comprising:
(a) providing a copper-dimer organometallic precursor;
(b) mixing the copper dimer organometallic precursor with a carbon nitride substrate to form a mixture; and
(c) heating the mixture to form a catalyst composite comprising a copper dimer having two copper atoms bridged by an oxygen atom, wherein each copper atom is coordinated to two nitrogen atoms of a carbon nitride substrate.

In certain embodiments, the copper-dimer organometallic precursor comprises an (oxalato) (bipyridine) copper (II) complex (Cu₂(bpy)₂(μ-ox)]Cl₂). In particular embodiments, the Cu₂(bpy)₂(μ-ox)]Cl₂ is prepared by reacting copper chloride (CuCl₂) with 2,2′,-bipyridine and oxalic acid.

In certain embodiments, the carbon nitride substrate comprises one or more of graphitic carbon nitride, α-carbon nitride, β-carbon nitride, cubic carbon nitride, pseudocubic carbon nitride, and combinations thereof. In particular embodiments, the carbon nitride substrate comprises graphitic carbon nitride. In more particular embodiments, the graphitic carbon nitride is prepared by calcination of urea.

In certain embodiments, the method for preparing the catalyst composite comprises heating the mixture from about 50° C. to about 250° C. for about 10 hours at a rate of about 2° C. min⁻¹.

In other embodiments, the presently disclosed subject matter provides a method for oxidizing methane, the method comprising contacting methane with the presently disclosed catalyst composite in the presence of an oxidizing agent.

In certain embodiments, the oxidizing agent comprises H₂O₂. In particular embodiments, the method comprises thermocatalytic oxidation of CH₄. In more particular embodiments, the methane, catalyst composite, and oxidizing agent are maintained at a predetermined pressure and temperature for a period of time.

In certain embodiments, the oxidizing agent comprises O₂. In particular embodiments, the method comprises photocatalytic oxidation of CH₄. In more particular embodiments, the methane, catalyst composite, and oxidizing agent are irradiated with visible light at a predetermined pressure and temperature for a period of time.

In certain embodiments, the predetermined pressure for either the thermocatalytic oxidation or photocatalytic oxidation of CH₄ is about 3 MPa, including about 1 MPa to about 5 MPa, including 1, 2, 3, 4, and 5 MPa. Likewise, in certain embodiments, the predetermined temperature is about 50° C., including about 35° C. to about 65° C., including 35, 36, 37, 38, 39, 40, 41, 42, 43, 44, 45, 47, 48, 49, 50, 51, 52, 53, 54, 55, 56, 57, 58, 59, 60, 61, 62, 63, 64, and 65° C. Further, in some embodiments, the period of time is between about 30 min to about 120 min, including about 30, 45, 60, 75, 90, 105, and 120 min.

In certain embodiments, the method of oxidizing CH₄ further comprises forming one or more methyl oxygenates. In particular embodiments, the one or more methyl oxygenates are selected from methanol (CH₃OH) and methyl hydroperoxide (CH₃OOH). In more particular embodiments, the method further comprises reducing the methyl hydroperoxide (CH₃OOH) to methanol (CH₃OH).

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Oxo Dicopper Anchored on Carbon Nitride for Selective Oxidation of Methane

### 1.1 Overview

Provided herein are dimeric copper centers supported on graphitic carbon nitride (denoted herein as Cu₂@C₃N₄) as advanced catalysts for the partial oxidation of CH₄. These catalysts are synthesized by immobilization of a copper-dimer organometallic complex on C₃N₄, with dicopper-oxo centers forming via mild calcinations. The derived Cu₂@C₃N₄ catalysts are characterized by combining scanning transmission electron microscopy (STEM), X-ray photoemission spectroscopy (XPS) and X-ray absorption spectroscopy (XAS), with the derived atomic structures of copper centers further confirmed by computational modeling based on density functional theory (DFT) calculations. The copper-dimer catalysts demonstrate high selectivity for partial oxidation of methane under both thermo- and photo-catalytic reaction conditions, with hydrogen peroxide (H₂O₂) and oxygen (O₂) being used as the oxidizer, respectively. In particular, the photocatalytic oxidation of CH₄ with O₂ achieves greater than 10% conversion and greater than 98% selectivity toward methyl oxygenates and a mass-specific activity of 1399.3 mmol gCu⁻¹ h⁻¹. Mechanistic studies reveal that the high reactivity of Cu₂@C₃N₄ for partial oxidation of methane can be ascribed to symphonic mechanisms among the bridging oxygen, the two copper sites and the semiconducting C₃N₄ substrate, which not only facilitate the heterolytic scission of the C—H bond, but also promote H₂O₂ and O₂ activation in thermo- and photo-catalysis, respectively.

### 1.2 Background

Selective conversion of methane (CH₄) into value-added chemicals represents a significant challenge for the efficient utilization of rising hydrocarbon sources. Direct, partial oxidation of methane to methyl oxygenates has received intensive attention in the recent years. Farrell and Linic, 2016; Hammond et al., 2012. Early studies used transition copper exchanged zeolites to catalyze the reaction between CH₄ and O₂, and employed a two-step chemical looping process to subsequentially activate O₂ and desorb the products. Woertink et al., 2009; Pappas et al., 2018; Groothaert et al., 2005. Despite the achievement of high selectivities, these reactions suffer from low CH₄ conversions (typically less than 0.03%) and reaction rates (less than 30 μmol g_{cata}⁻¹ h⁻¹). Narsimhan et al., 2016; Dinh et al., 2019; Koishybay and Shantz, 2020.

Later on, partial oxidation of methane in a single step was demonstrated by using non-O₂ oxidizers, such as oleum, Palkovits et al., 2009), selenic acid, Jones et al., 2004) and H₂O₂, Shan et al., 2017; Sushkevich et al., 2017; Grundner et al., 2015, but the cost associated with these oxidizing agents restricts their practical implementations. Agarwal et al., 2017. More recent efforts have thus turned to in-situ generation of H₂O₂ from O₂ by using selective oxygen reduction catalysts, such as Au—Pd containing zeolites. Jin et al., 2020.

Alternatively, photo-excitation using visible light is proposed to be advantageous with near-room temperature activation of CH₄, thereby mitigating the concern of over oxidation to form CO₂ upon heating. Song et al., 2019a; Song et al., 2019b. The reported photocatalytic oxidation of methane, however, is still limited by relatively low methane conversions (less than 1%) and productivities (between about 0.001 to about 150 mmol g_{cata}⁻¹ h⁻¹), Song et al., 2019b, because the commonly used photocatalysts have quite large bandgaps (e.g., approximately 3.2 eV for TiO₂, Song et al., 2020, and approximately 3.4 eV for ZnO, Song et al., 2019a) and may only activate methane via the Fenton or homolytic mechanisms that have relatively sluggish kinetics. Szecsenyi et al., 2018a.

In this respect, graphitic carbon nitride (g-C₃N₄) represents a promising photocatalytic substrate with a modest band gap in the range of 2.7-2.9 eV. Wen et al., 2017; Xu and Gao, 2012; Su et al., 2010. Its abundant nitrogen sites have been shown to be capable of anchor atomically dispersed transition metal sites. Shi et al., 2020. It thus becomes interesting to investigate the potential coordination of active Cu sites on g-C₃N₄ and examine their synergies in the partial oxidation of methane.

### 1.3 Scope

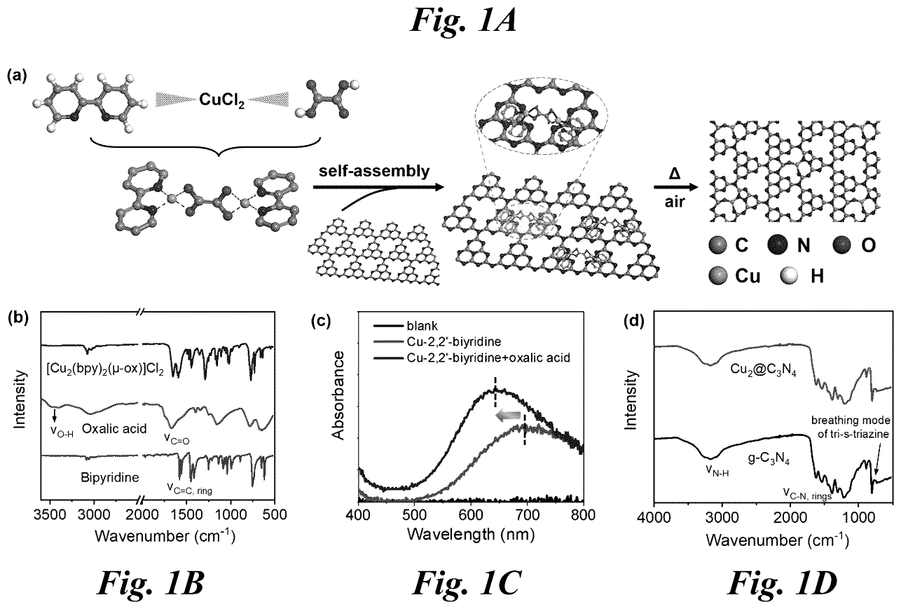

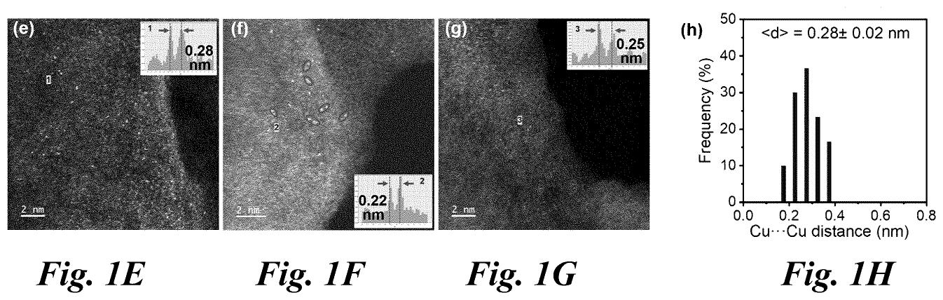

The presently disclosed subject matter demonstrates Cu₂@C₃N₄ as highly efficient catalysts for the partial oxidation of methane. The dimeric copper catalysts were synthesized by supporting an (oxalato) (bipyridine) copper (II) complex, [Cu₂(bpy)₂(μ-ox)]C₁₂, on g-C₃N₄ and then applying a mild thermal treatment in air (FIG. 1a). The derived catalysts contained dicopper-oxo centers anchoring on g-C₃N₄ via four Cu—N bonds (two for each copper atom), as characterized by using STEM, XPS and XAS, and also confirmed with atomistic simulations. The obtained copper-dimer catalysts were first evaluated for thermal oxidation of methane using H₂O₂ as the oxidizer, and then further applied for photocatalytic oxidation of methane with O₂. Mechanisms governing the observed catalytic enhancements toward selective oxidation of methane were interpreted via combining computational simulation of the reaction pathways, spin-trapping EPR analysis of possible radical intermediates and in situ XPS measurements under light irradiation.

### 1.4 Results and Discussion

### 1.4.1 Synthesis and Characterization of Cu₂@C₃N₄

The copper-dimer precursor [Cu₂(bpy)₂(μ-ox)]Cl₂ was first prepared by a complexation reaction of copper chloride (CuCl₂), 2,2,-bipyridine and oxalic acid. Reinoso et al., 2003. The g-C₃N₄ substrate was grown by calcination of urea at 550° C. Martin et al., 2014. Cu₂@C₃N₄ catalysts were synthesized by self-assembly of the dimeric copper complex on g-C₃N₄, Zhao et al., 2018, and then treating the mixture in air at 250° C. to immobilize the copper species (FIG. 1a). The loading of Cu was determined to be 0.35 wt % by using inductively coupled plasma mass spectrometry (ICP-MS).

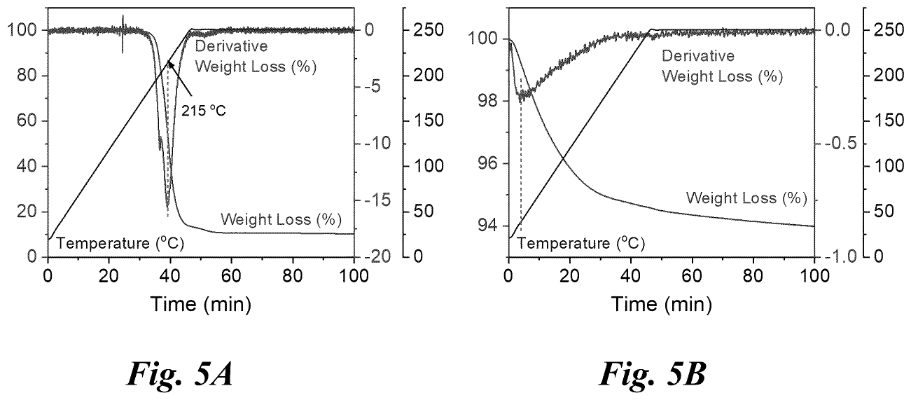

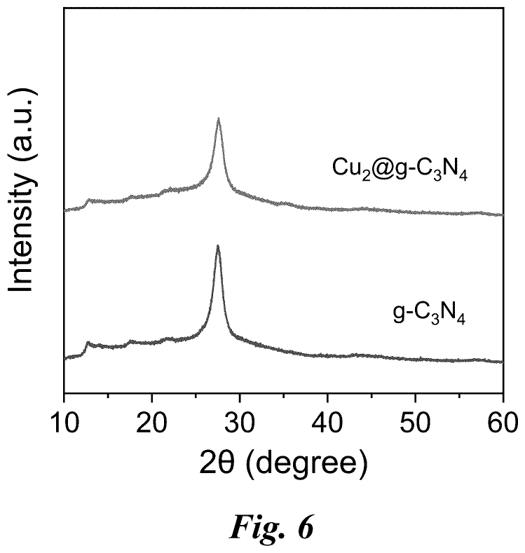

The complexation of pyridine, Cu²⁺ and oxalate (C₂O₄²⁻) to form an organometallic compound was confirmed by using Fourier transform infrared spectroscopy (FTIR). The hydroxyl (O—H) and carbonyl (C═O) stretching features around 3,450 cm⁻¹ and 1,670 cm⁻¹, respectively, which are associated with oxalic acid disappeared after the reaction. This disappearance was accompanied with the blue shift of the characteristic band (attributed to the asymmetric stretching of the pyridyl ring) of 2,2′-pyridine at ca. 1,580 cm⁻¹ to ca. 1,650 cm⁻¹, a consequence of its chelation with Cu₂+ (FIG. 1b). Gerasimova and Katsyuba, 2013. Correspondingly, the d-d transition of Cu²⁺ at about 650 nm to about 700 nm had a blue shift of 48 nm in the ultraviolet-visible diffuse reflectance spectroscopy (UV-vis DRS) patterns (FIG. 1c). The FTIR spectra of g-C₃N₄ and Cu₂@C₃N₄ exhibited the stretching vibration modes characteristic of the —NH group around 3,180 cm⁻¹, C—N heterocycles in the wavelength range of 1,100-1,650 cm⁻¹ and the breathing mode of tri-s-triazine units at 810 cm⁻¹ (FIG. 1d). Compared to [Cu₂(bpy)₂(μ-ox)]Cl₂, Cu₂@C₃N₄ presented no infrared features associated with the dimeric copper complex, indicating the complete removal of organic ligands during the immobilization process. This observation was further confirmed with thermogravimetric analysis (TGA) (FIG. 5). The [Cu₂(bpy) 2 (μ-ox)]Cl₂/g-C₃N₄ mixture lost approximately 6% of its initial weight upon annealing in air at up to 250° C., which is close to the expectation estimated based on the ligand content (approximately 80 wt %) of [Cu₂(bpy)₂(μ-ox)]Cl₂ and its ratio relative to the carbon nitride substrate (approximately 8%) used in the synthesis. X-ray diffraction (XRD) patterns collected for the Cu₂@C₃N₄ catalysts only show the (001) and (002) peaks associated with g-C₃N₄, with the absence of copper metal or oxide features indicating the highly dispersed nature of copper species (FIG. 6).

Atomic structures of the dimeric copper moieties were resolved by using aberration correction high-angle annular dark-field scanning transmission electron microscopy (HAADF-STEM) imaging (FIG. 1e, FIG. 1f, and FIG. 1g). The collected STEM images exhibit a large number of adjacent, paired bright dots (labeled with red circles, less than 0.35 nm in size for each dot) distributed on a substrate of lower contrasts. These small bright dots can be attributed to atomically dispersed Cu considering their much higher Z contrast (M=65 for Cu) than C₃N₄ (M=12 or 14). Line-profile scanning for approximately 100 pairs of such bright dots give an average distance of 2.8 (+0.2 Å) (FIG. 1h). This average distance is much shorter than the value (5.2 Å) for the two copper atoms within [Cu₂(bpy)₂(μ-ox)]Cl₂, again confirming the reconstruction and condensation of the copper-dimer moieties as a result of the removal of organic ligands in the synthesis.

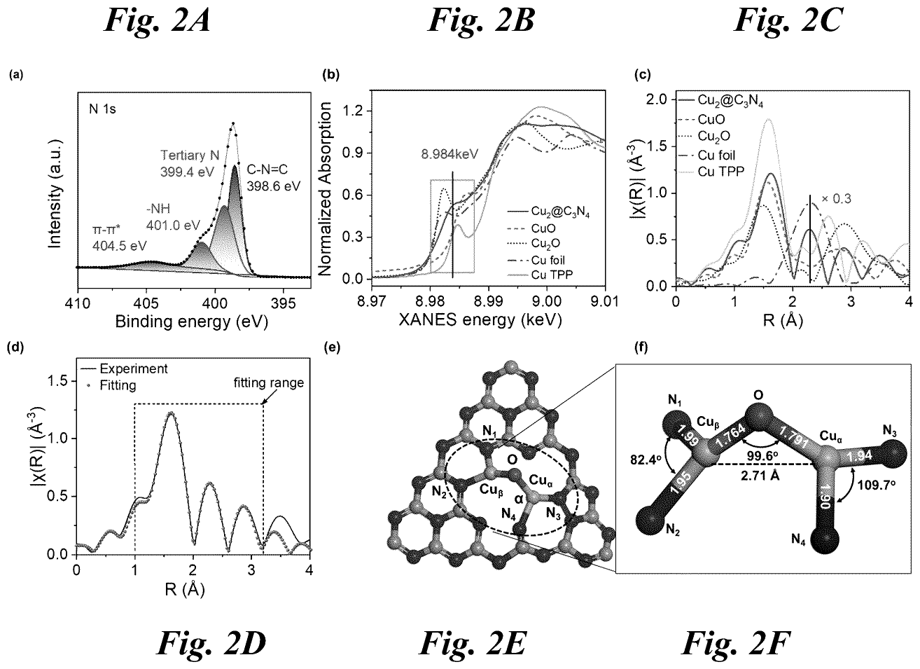

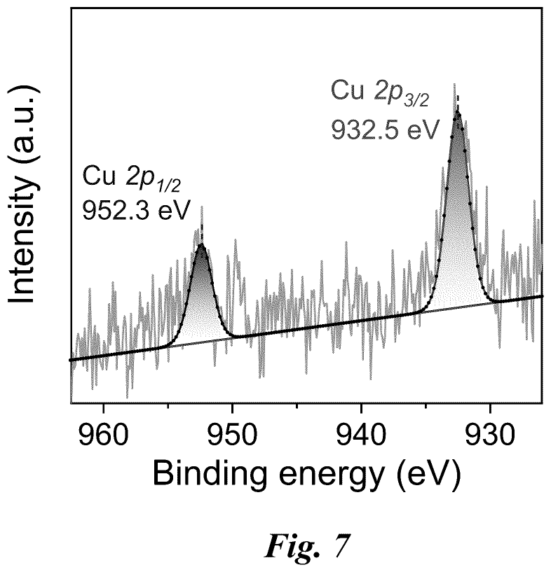

The chemical nature of the Cu dimers in Cu₂@C₃N₄ was probed by using X-ray photoelectron spectroscopy (XPS) and X-ray absorption spectroscopy (XAS). The N 1s XPS spectrum exhibits a broad feature with the binding energy ranging from 397 eV to 408 eV (FIG. 2a). This feature can be deconvoluted into four peaks centered at 398.6 eV, 399.4 eV, 401.0 eV, and 404.5 eV, which can be assigned to pyridinic (C—N═C), tertiary (—N<), pyrrolic (—NH) nitrogen and π-π* transition of C═N or uncondensed terminal amine groups in g-C₃N₄, respectively. Mane et al., 2017; Chen et al., 2017. The Cu 2p spectrum shows two peaks at 932.5 eV and 952.3 eV, which are characteristic of Cu(I) or Cu⁰ (FIG. 7). Fox et al., 2008.

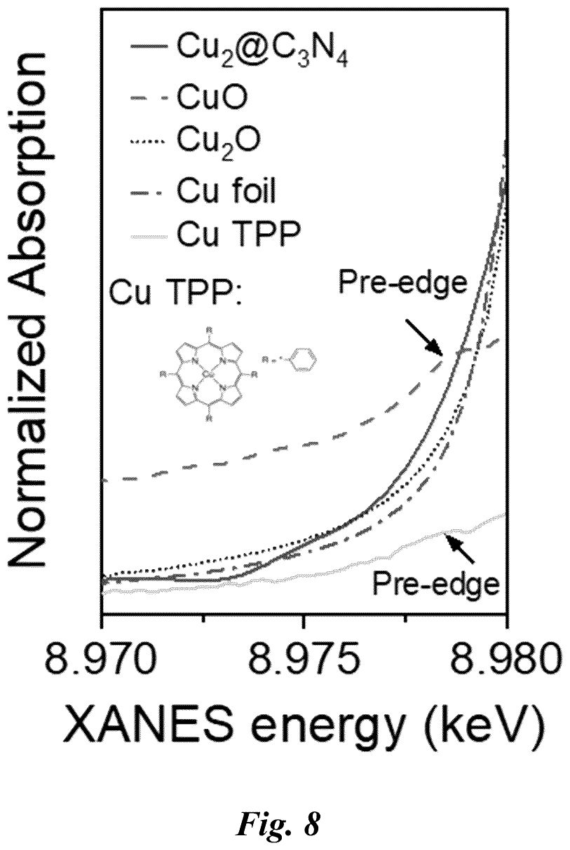

The XPS analysis, however, as well as the corresponding Auger electron spectrum (AES), was unable to explicitly determine the oxidation state of Cu due to the reduced signal-to-noise ratio associated with the low copper content in the catalysts. The copper oxidation state in Cu₂@C₃N₄ was better resolved by using X-ray Absorption Near Edge Spectroscopy (XANES) (FIG. 2b). The Cu K-edge spectrum exhibits a pre-edge transition at 8,984 eV, which falls between the peaks associated with Cu₂₀ (8,983 eV) and CuO (8,986 eV). This indicates an intermediate oxidation state between +1 and +2 for Cu in Cu₂@C₃N₄. Noticeably, these results do not support a mixture of Cu (I) and Cu(II), as the 1s-+3d transition at 8,977 eV, a feature characteristic of Cu²⁺ (as shown for the references CuO and copper tetraphenylporphyrin (Cu-TPP) in FIG. 8), Fox et al., 2008, is absent in the spectrum of Cu₂@C₃N₄. The partial oxidation state (between +1 and +2) of Cu within the copper dimers supported on g-C₃N₄ can be viewed as a result of the semiconducting nature of the substrate. This characteristic is distinguished from the extensively studied copper-exchanged zeolites, in which the dicopper-oxo centers ([Cu—O—Cu]²⁺) anchor on the Al sites with localized negative charges and have an oxidation state of +2 for both Cu atoms. Groothaert et al., 2005; Xie et al., 2021; Tsai et al., 2014.

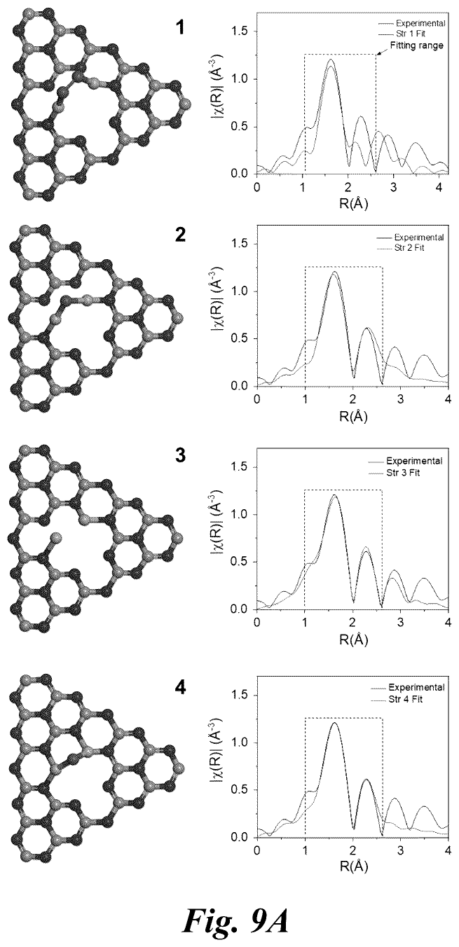

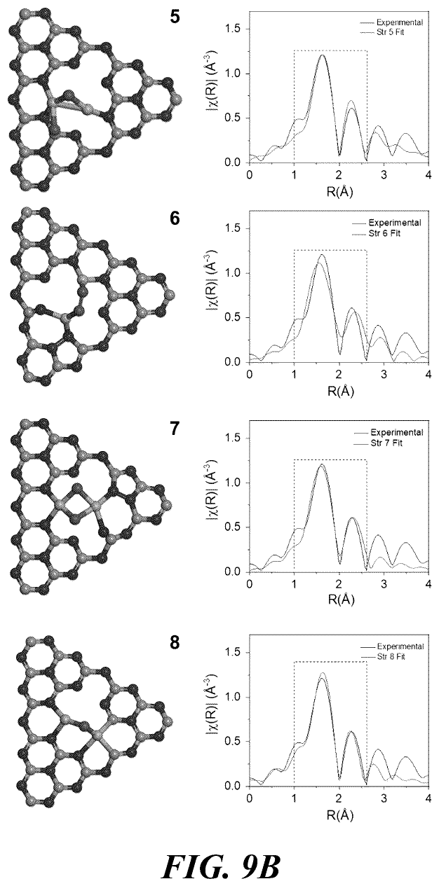

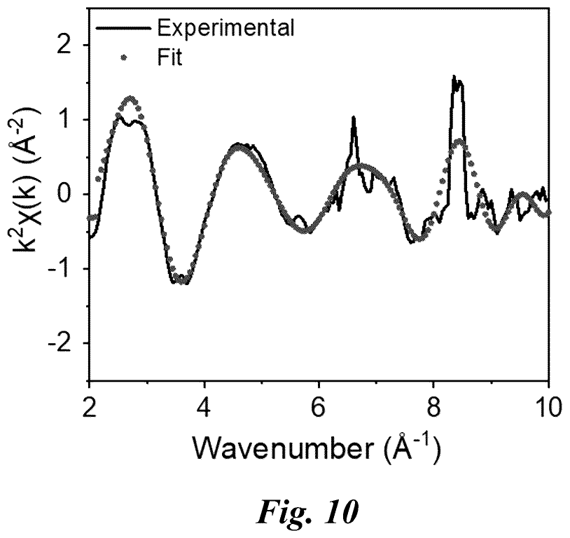

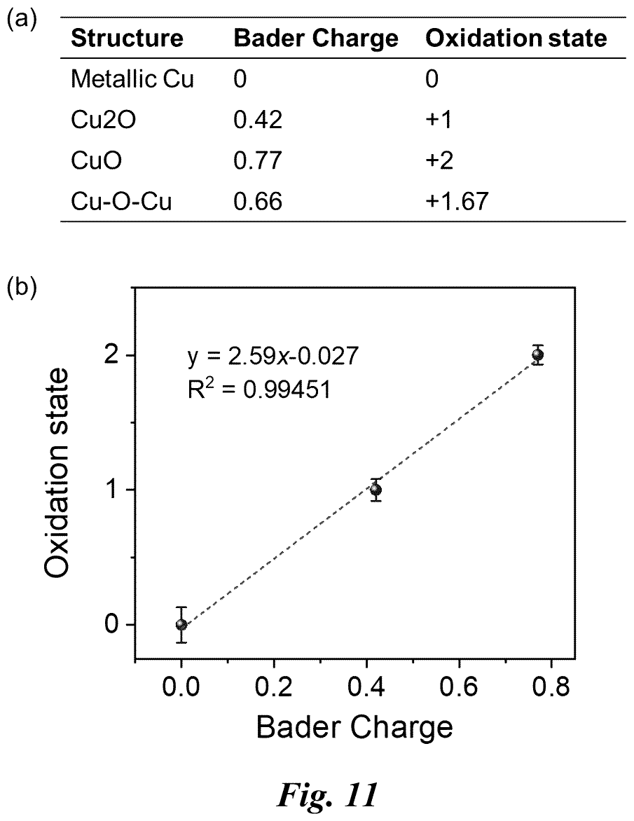

The atomic structure of the Cu dimers was resolved by combining extended X-ray absorption fine structure (EXAFS) analysis and atomistic modeling based on DFT calculations. FIG. 2c compares the k²-weighted Cu K edge EXAFS spectra for Cu₂@C₃N₄, Cu foil, Cu₂O, CuO and Cu-TPP (with single-atom Cu²⁺ coordinating to four pyrrolic N, FIG. 8). The Cu₂@C₃N₄ catalyst exhibits first-shell scattering at 1.62 Å in R space (prior to phase correction), which is proximate to the values, 1.59 and 1.55 Å, found for Cu-TPP and CuO, respectively. This characteristic is distinct from that for Cu₂O and Cu, the first-shell scattering of which locates at 1.50 and 2.30 Å, respectively. From these observations, the primary scattering pair at 1.62 Å in the R-space spectrum of Cu₂@C₃N₄ was tentatively assigned to be Cu—N or Cu—O bonding. To fit the EXAFS spectrum, a total of 9 possible Cu-dimer configurations was postulated, based on which DFT calculations were performed to relax the structures to determine bonding distances and angles (FIG. 9 and Table 1). Considering the presence of minor Cu monomers observed in the STEM images (FIG. 1e, FIG. 1f, and FIG. 1g), a linear combination of Cu monomers and dimers was applied to fit the EXAFS spectrum. Various possible Cu-dimer configurations have been considered, with the corresponding EXAFS spectra compared to the experimental results to identify the best fit (FIG. 2d, FIG. 2e; also see FIG. 10 and FIG. 11). It is estimated that 72.4% of the Cu atoms are in the dimeric configuration, close to the value (approximately 70%) derived from statistical analysis of the STEM images. The determined copper-dimer structure comprises two Cu atoms bridged by an O atom, with the Cu—O bonds having lengths of 1.76 Å and 1.79 Å and an included angle (∠Cu—O—Cu) of 99.6° (FIG. 2f, Table 1). Each Cu atom is coordinated to two N atoms on the C₃N₄ framework, with the bonding distance varying from 1.90 to 1.99 Å and the bonding angle (∠N—Cu—N) being 82° for Cu_{α} and 110° for Cu_{β} (FIG. 2f). Noticeably, the identified configuration best fitted to the EXAFS spectrum also has the lowest (most negative) formation energy among the various configurations, in line with the expectation for stable atomic structures in the real catalysts (Table 2). The combined EXAFS analysis and DFT calculations resolved the Cu—Cu distance in the Cu dimers to be approximately 2.71 Å (Table 1), which is in agreement with the average Cu—Cu distance measured from the STEM images (FIG. 1h). It is noted that this value is much smaller than the Cu—Cu distance (4.10 Å) associated with the dicopper-oxo center ([Cu—O—Cu]²⁺) in Cu-ZSM-5. Tsai et al., 2014. Furthermore, Bader charge analysis based on DFT calculation show that the Cu atoms in the Cu dimers have oxidation states of +1.63 and +1.72 (FIG. 11), resembling the results derived from XANES spectra (see the above discussion for FIG. 2b).

TABLE 1
Structural parameters being used to fit the EXAFS spectrum for
Cu₂@C₃N₄ with consideration of both monomeric and dimeric Cu sites.

| Scattering path | CN | Distance (Å) | σ²(Å²) | R-factor |
|---|---|---|---|---|
| Cu—O | 1.25 ± 0.20 | 1.77 ± 0.01 | 0.0056 ± 0.0006 | 0.009 ± 0.001 |
| Cu—N (Cu dimer) | 1.84 ± 0.29 | 1.99 ± 0.01 | | |
| Cu—N (Cu monomer) | 2.42 ± 0.32 | 1.98 ± 0.01 | | |
| Cu—Cu | 0.88 ± 0.15 | 2.71 ± 0.01 | 0.0038 ± 0.0004 | |

TABLE 2
The formation energies of different structures
studied for the optimization of Cu dimer.

| Structures | ΔE (eV) |
|---|---|
| 1 | −8.09 |
| 2 | −8.74 |
| 3 | −5.58 |
| 4 | −8.09 |
| 5 | −8.97 |
| 6 | 2.62 |
| 7 | −6.72 |
| 8 | −9.03 |
| 9 | −12.74 |

To calculate the formation energies of potential Cu dimer structures, the following equation was used:

E(formation energy)=E(System)−E(Substrate)−α×E(Cu)−b×0·EO₂)

Here, E(System) represents the total free energy of the system, E(Substrate), E(Cu) and E(O₂) represent the free energies of the support, Cu atoms and oxygen gas, and a and b are the numbers of Cu and O atoms involved in the considered structure.

From the above discussion, it can be seen that the dimeric copper centers in Cu₂@C₃N₄ have distinct atomic structures and electronic chemical properties compared to their counterparts confined in zeolites. Without wishing to be bound to any one particular theory, it is thought that their non-integer oxidation state (intermediate between +1 and +2) and reduced cluster size (smaller Cu—Cu distance as compared to Cu-ZSM-5) would lend them exquisite catalytic performance for selective oxidation of methane. Xie et al., 2021.

### 1.4.2 Thermocatalytic Oxidation of Methane with H₂O₂

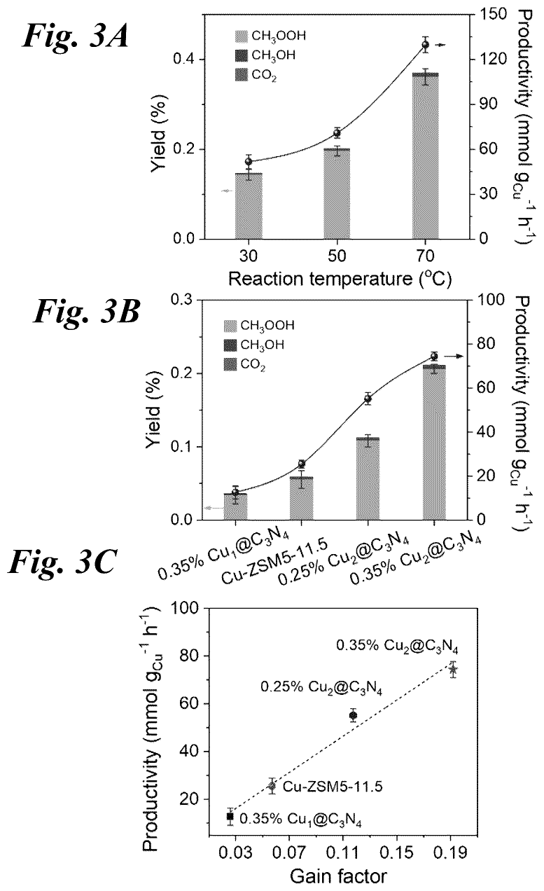

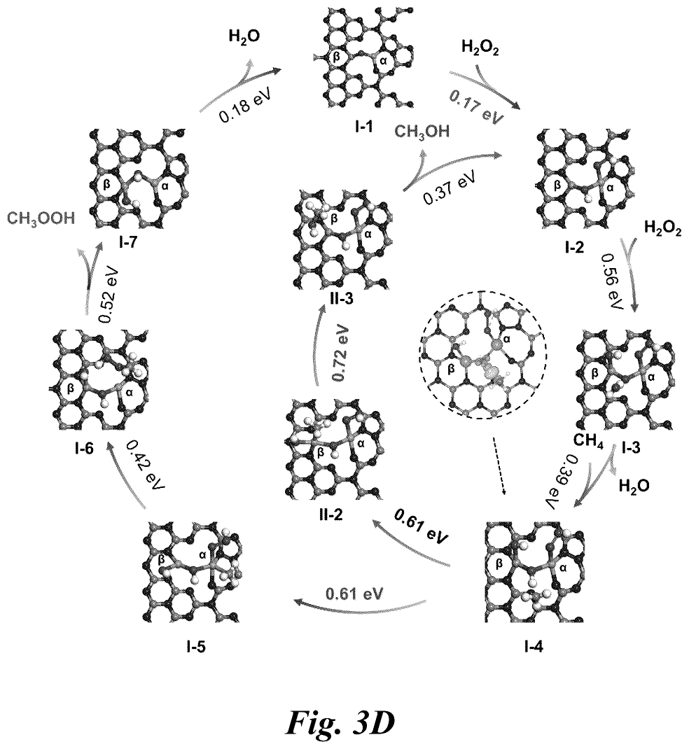

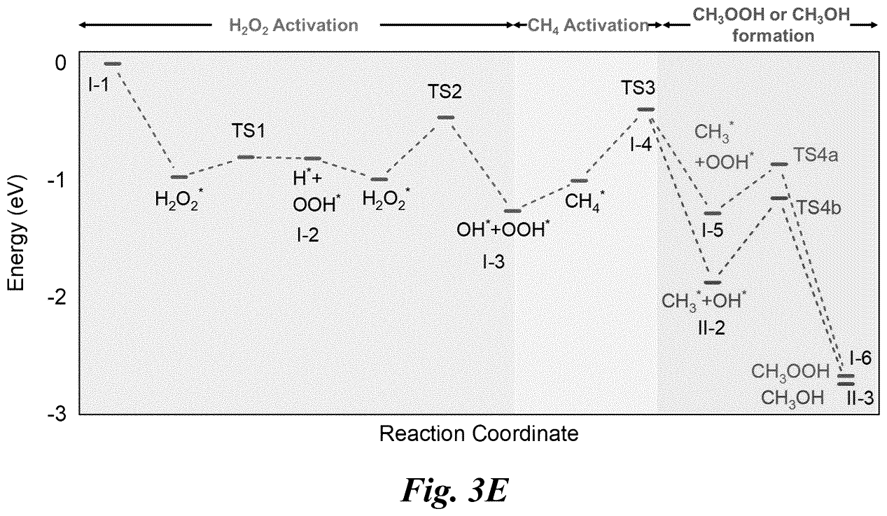

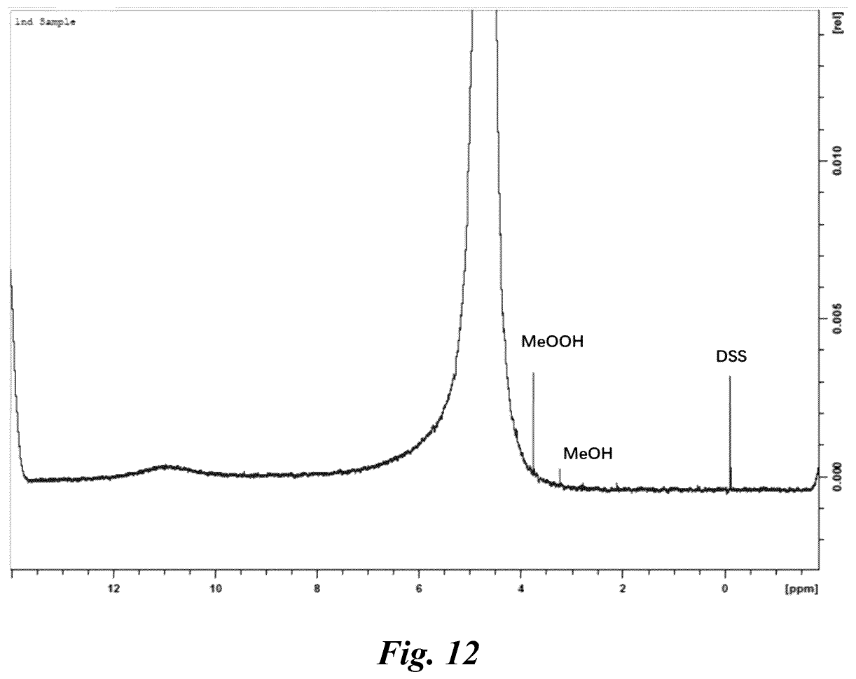

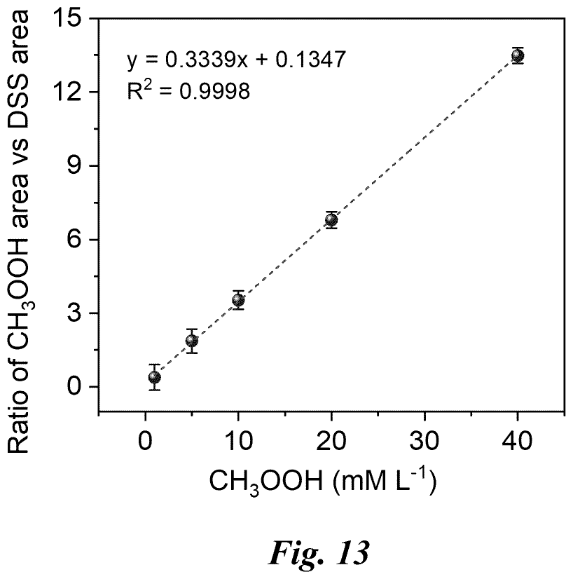

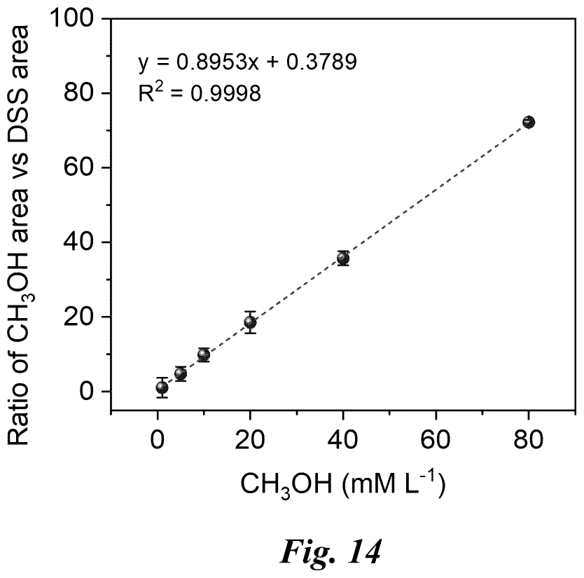

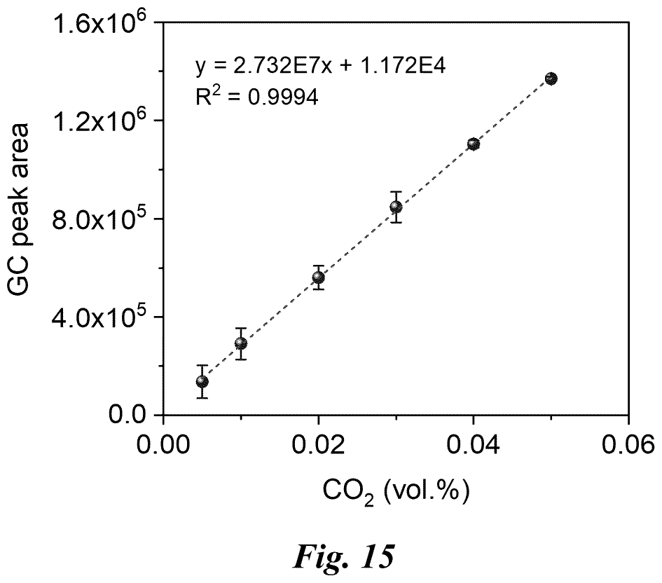

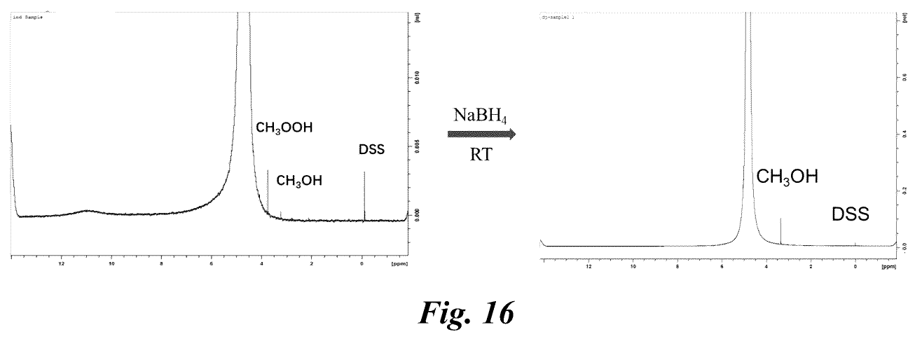

The Cu₂@C₃N₄ catalysts were first evaluated for the thermocatalytic oxidation of CH₄ (FIG. 12, FIG. 13, FIG. 14, FIG. 15, and FIG. 16). This evaluation was conducted using a continuous stirred-tank reactor (CSTR) filled with 0.2 mM of H₂O₂ and 0.1 MPa of CH₄ (see 1.5 Methods). Methyl oxygenates (CH₃OH and CH₃OOH) were found to be the primary products, with the yield achieving 0.14% within 30 min of reaction at 30° C. (FIG. 3a). As previously reported, the generated CH₃OOH can be facilely reduced to CH₃OH under ambient conditions (FIG. 16). Agarwal et al., 2017; Ab Rahim et al., 2013.

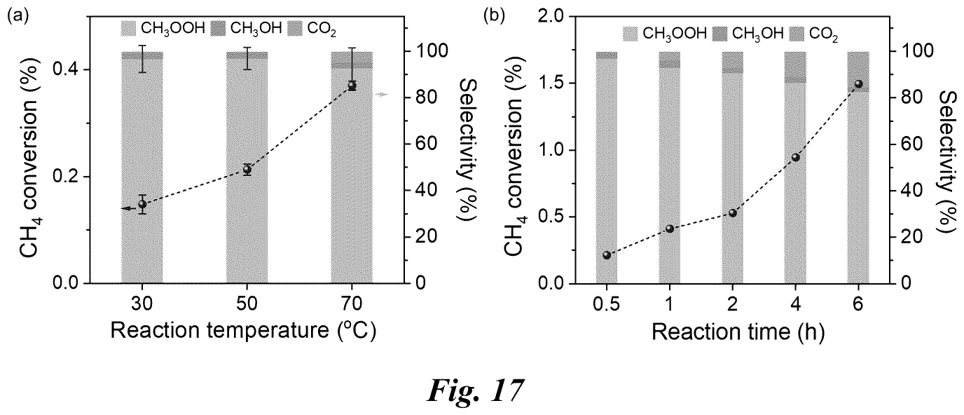

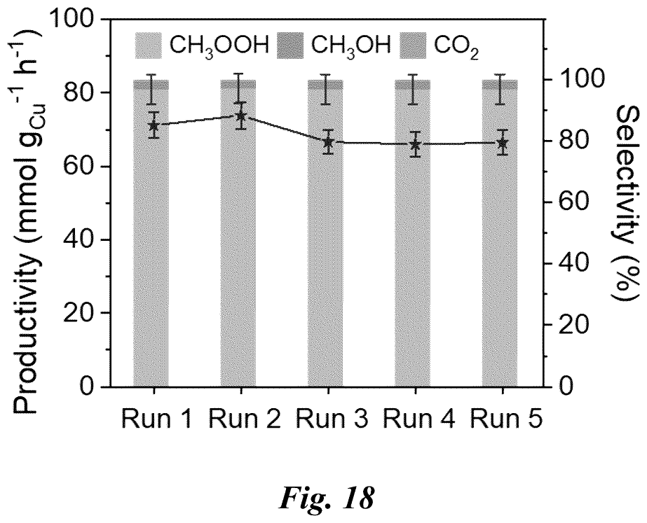

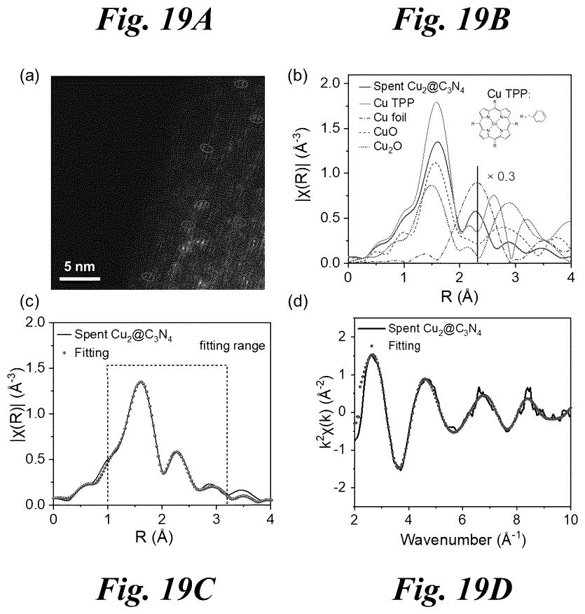

The yield of methyl oxygenates increased to 0.37% at 70° C., corresponding to the increase of productivity from 51.6 to 129.7 mmol g_{Cu}⁻¹ h⁻¹. Albeit the increase of reaction rate, the rise of reaction temperature is accompanied with the increase of CO₂ selectivity from 0.8% at 30° C. to 5.0% at 70° C. (FIG. 17). Similarly, elongated operations also led to the yield of more CO₂ (FIG. 17). The cyclability tests showed that the Cu₂@C₃N₄ catalyst was stable throughout the methane oxidation reaction with H₂O₂. In five consecutive measurements by refilling CH₄ and H₂O₂, the Cu₂@C₃N₄ catalyst exhibited indiscernible change in reactivity and product distribution, with the productivity of methyl oxygenates found to be consistent at approximately 70 mmol g_{Cu}⁻¹ h⁻¹ at 50° C. (FIG. 18). Furthermore, the atomic structure of dicopper-oxo centers was confirmed to remain intact after reaction by performing HADDF-STEM imaging and EXAFS analysis on the spent Cu₂@C₃N₄ catalyst after 6 h of reaction (FIG. 19 and Table 3).

TABLE 3
Structural parameters being used to fit the EXAFS spectrum
of the spent Cu₂@C₃N₄ catalyst after 6 h of reaction.

| Scattering path | CN | Distance (Å) | σ²(Å²) | R-factor |
|---|---|---|---|---|
| Cu—O | 1.35 ± 0.24 | 1.77 ± 0.01 | 0.0049 ± 0.0005 | 0.008 ± 0.001 |
| Cu—N (dimer) | 1.91 ± 0.33 | 1.99 ± 0.01 | | |
| Cu—N (single atom) | 2.42 ± 0.32 | 1.98 ± 0.01 | | |
| Cu—Cu | 0.81 ± 0.14 | 2.71 ± 0.01 | 0.0043 ± 0.0004 | |

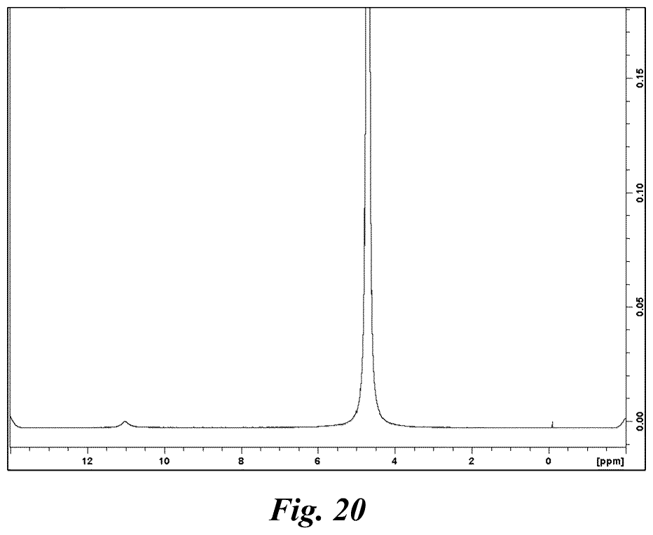

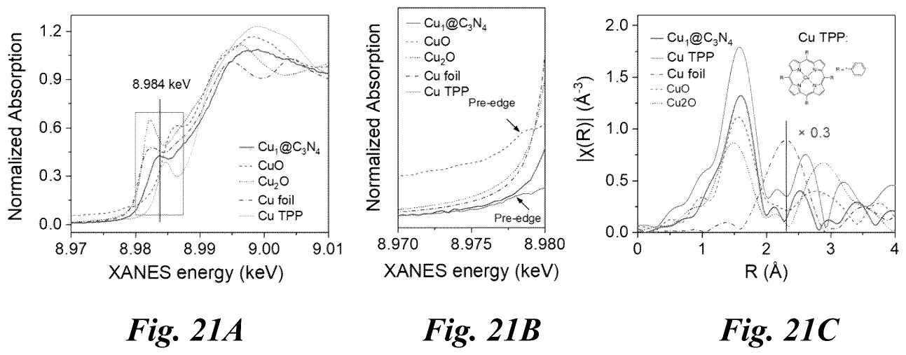

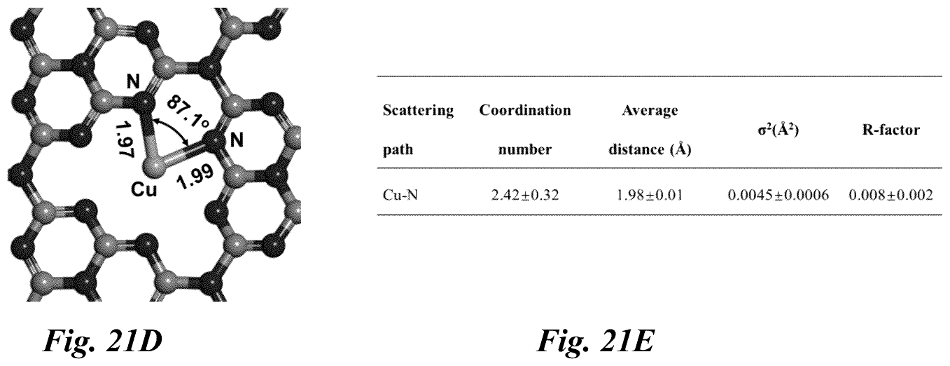

Considering that the bare g-C₃N₄ substrate is inactive for CH₄ oxidation (FIG. 20), the dicopper-oxo centers can be identified as the active sites in Cu₂@C₃N₄. Considering that monomeric Cu in copper exchanged zeolites, Woertink et al., 2009; Kulkarni et al., 2018; Yashnik et al., 2020, or metal-organic frameworks, Zheng et al., 2019, also has been discussed to be active for CH₄ oxidation, comparative studies were performed on a single-atom control (Cu₁@C₃N₄) using the same g-C₃N₄ substrate. This catalyst was prepared by using a vapor-migration strategy, Qu et al., 2018, with the Cu loading also controlled to be at approximately 0.35 wt %, with the single-atom dispersion confirmed by using XAS (FIG. 21).

Catalytic studies showed that Cu₁@C₃N₄ was barely active for methane oxidation, delivering a yield of only 0.03% (versus 0.2% by Cu₂@C₃N₄) for methyl oxygenates at 50° C. (FIG. 3b). The low reactivity of Cu₁@C₃N₄ indicates that Cu monomers, if present in the Cu₂@C₃N₄ catalysts, would not make significant contributions to the observed high methane partial oxidation activity, and also underlines the necessity of having dicopper-oxo centers for catalyzing the partial oxidation of methane. The activity of Cu₂@C₃N₄ also is substantially enhanced as compared to copper-exchanged zeolites. A comparative study of Cu-ZSM5 with a Si/Al ratio of 11.5 and full exchange (Cu/Al approximately 0.51) using similar reaction conditions only delivered a productivity of 25.5 mmol g_{Cu}⁻¹ h⁻¹ for methyl oxygenates at 50° C., as compared to 74.4 mmol g_{Cu}⁻¹ h⁻¹ for Cu₂@C₃N₄ at this temperature (FIG. 3b). Note that the copper species in this Cu-ZSM-5 catalyst also is predominantly present in the form of dicopper-oxo centers, Xie et al., 2021, similar to that identified in Cu₂@C₃N₄ (as shown in FIG. 2f). These results indicate that the dimeric Cu supported on carbon nitride is much more reactive for the oxidation of methane with H₂O₂ than their counterparts confined in zeolites.

In the partial oxidation of methane with H₂O₂, the efficiency of utilizing the peroxide oxidizer (instead of producing O₂ through a disproportionation reaction) is an important metric for evaluating the performance of catalysts. Agarwal et al., 2017; Ravi et al., 2019. This metric is usually assessed by comparing the “gain factor” that is defined as the molar ratio between the produced methyl oxygenates (CH₃OH and CH₃OOH) and the consumed H₂O₂. Agarwal et al., 2017.

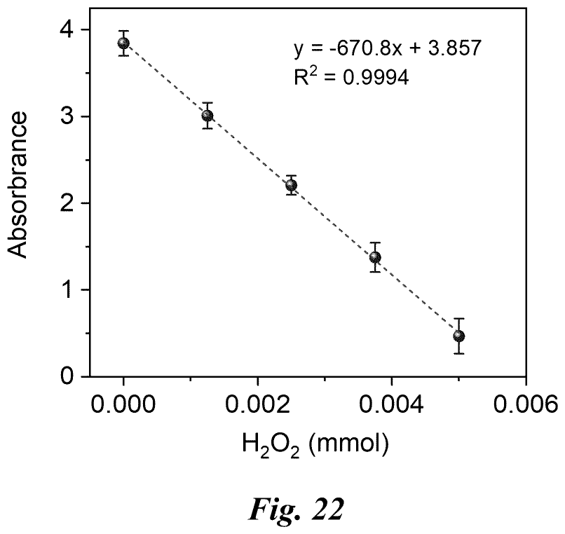

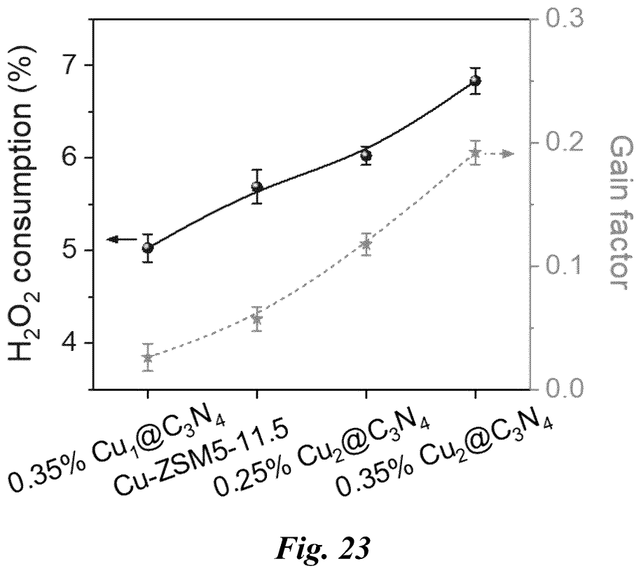

Post-reaction titration of the concentration of residual hydrogen peroxide using cerium sulfate, Lu et al., 2018, (FIG. 22) showed that the Cu₂@C₃N₄ catalyst had a gain factor of 0.19 (FIG. 3c and FIG. 23). In comparison, the gain factor was determined to be only 0.03 and 0.06 for Cu₁@C₃N₄ and Cu-ZSM5-11.5, respectively. It is interesting that the gain factor exhibited dependence on the copper loading in the dimer catalyst. A Cu₂@C₃N₄ catalyst of reduced loading (0.25 wt %) had a gain factor of 0.12, which is lower than that for the normal catalyst with 0.35 wt % of copper. Moreover, correlation between the productivity of methyl oxygenates and the gain factor gives rise to a linear relationship, underscoring its meaning of describing the reactivity between methane and H₂O₂ on a given catalyst (FIG. 3c). Agarwal et al., 2017; Xing et al., 2021.

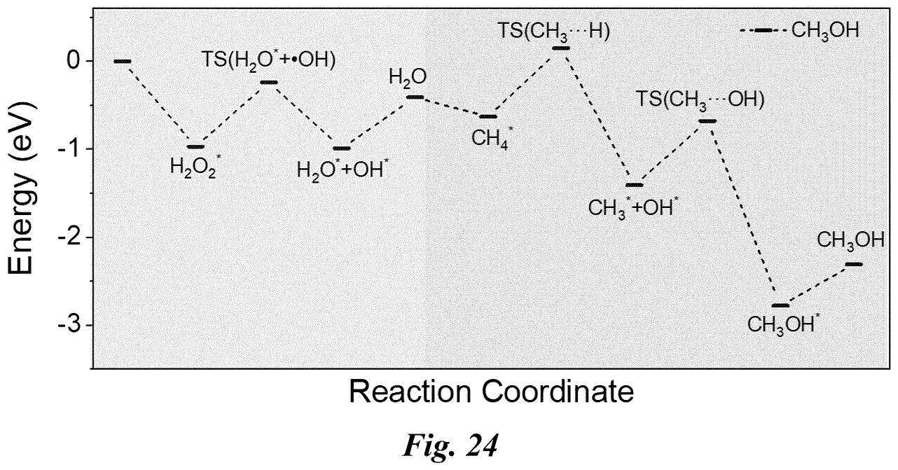

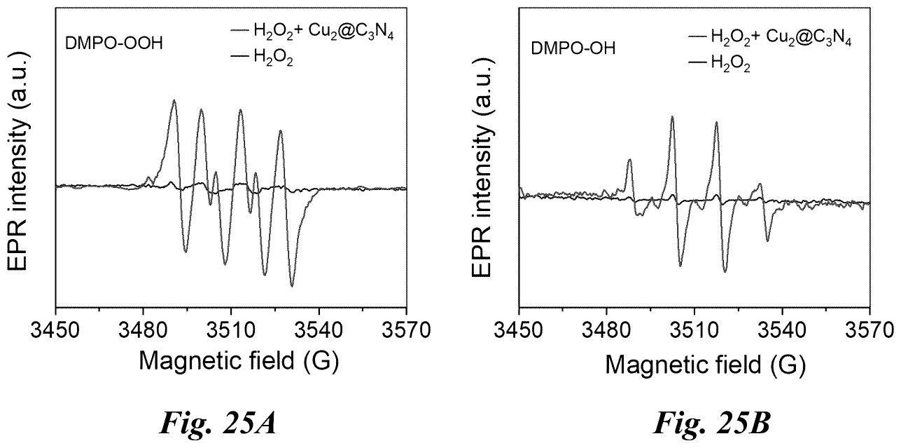

To understand the enhanced reactivity of Cu₂@C₃N₄ for methane partial oxidation, DFT calculations were performed to simulate the reaction pathways on the dicopper-oxo centers (FIG. 3d, FIG. 3e; also see FIG. 24 and Table 4). It is predicted that the reaction starts with sequential activation of two H₂O₂ molecules on the copper-dimer centers through radical mechanisms. Szecsenyi et al., 2018a; Szecsenyi et al., 2018b; Osadchii et al., 2018. The first hydrogen peroxide molecule is dissociated via H₂O₂→·OOH+*H, where the hydrogen adsorbs on the bridging oxygen and the ·OOH radical migrates onto Cu_{α} to become a peroxyl (*OOH) adsorbate. The second hydrogen peroxide undergoes H₂O₂→·OH+*OH with the hydroxyl group adsorbing on Cu_{β} and the ·OH radical recombines with the *H on the bridging oxygen site to form a H₂O molecule. The involvement of ·OOH and ·OH in H₂O₂ activation was corroborated by the observation of these radicals in the electron paramagnetic resonance (EPR) spectroscopic studies by using 5,5′-dimethyl-1-pyrroline-N-oxide (DMPO) as the radical trap (FIG. 25). Schneider et al., 2020.

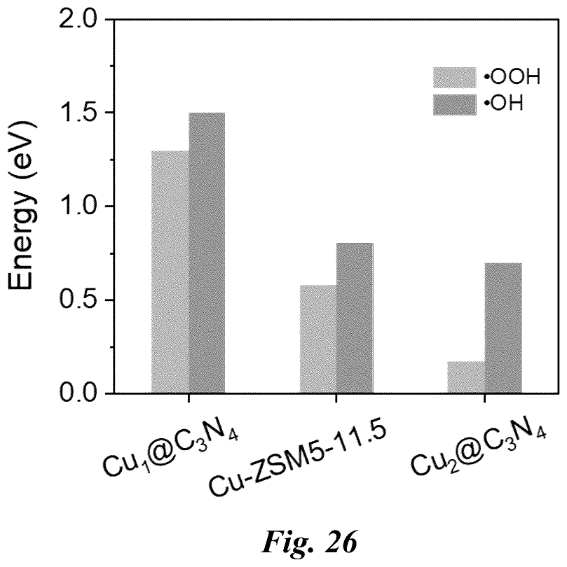

The generation of radicals is the rate limiting factor in both cases of H₂O₂ activation, which is predicted to have a kinetic barrier of 0.17 (for ·OOH) or 0.56 (for ·OH) eV. Noticeably, these barriers are substantially lower than the corresponding values found for the single-atom Cu sites (1.3 and 1.5 eV, FIG. 26) and the dicopper-oxo centers confined in zeolites (0.58 and 0.81 eV in Cu-ZSM-5), Hammond et al., 2012; Hori et al., 2018, in line with the higher gain factor and enhanced utilization of H₂O₂ as observed on the Cu₂@C₃N₄ catalysts (FIG. 3c). The enhanced H₂O₂ activation on Cu₂@C₃N₄ could be ascribed to the π-conjugated heterocyclic rings and the semiconducting nature of the C₃N₄ substrate, which is known for accommodation of charge transfer and able to supply electrons to the dicopper-oxo center for stabilization of the oxygenated adsorbates. Zhou et al., 2019; Xiao et al., 2020; Guo et al., 2021; Tong et al., 2018. The C₃N₄ supported Cu dimers are thus believed to be more advantageous than their zeolitic counterparts for catalyzing the redox chemistries being examined here.

TABLE 4
Energy barriers and enthalpies for elementary steps
during the thermal catalytic CH₄ selective oxidation by H₂O₂ to
CH₃OH on fresh Cu dimer of Cu₂@C₃N₄.

| | Elementary steps | Eₐ (eV) | ΔH (eV) |
|---|---|---|---|
| R1 | H₂O₂ + * → H₂O₂* | 0.00 | −0.97 |
| R2 | H₂O₂* + H*→OH* + H₂O* | 0.73 | −0.02 |
| R3 | H₂O*→H₂O + * | 0.58 | 0.58 |
| R4 | CH₄ + *→CH₄* | 0.00 | −0.22 |
| R5 | CH₄*→CH₃* + H* | 0.76 | −0.78 |
| R6 | CH₃* + OH*→CH₃OH* | 0.73 | −1.37 |
| R7 | CH₃OH*→CH₃OH + * | 0.47 | 0.47 |

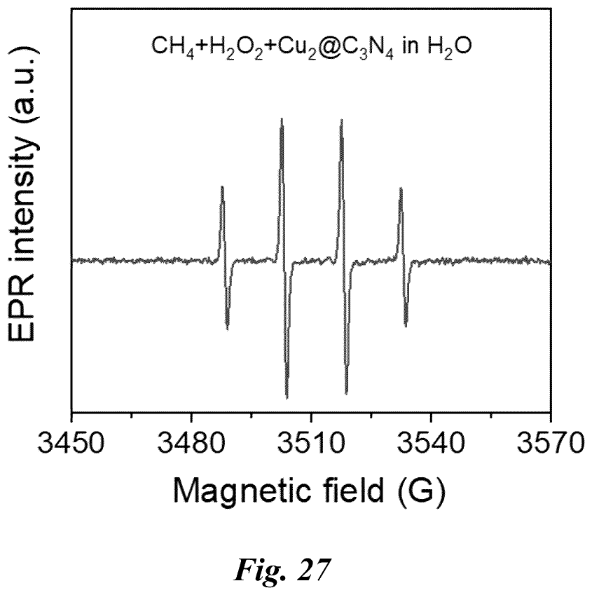

Following the activation of H₂O₂, methane is introduced to the dicopper oxo center with one of the C—H bond attacked by the bridging oxygen (FIG. 3d and FIG. 3e). This C—H bond dissociation has a modest energy barrier of 0.61 eV (vs. approximately 0.71 eV in the case of Cu-ZSM-5). Pappas et al., 2018; Kulkarni et al., 2018; Yashnik et al., 2020. While the generated H adsorbs on the bridging oxygen, the methyl group migrates on to the adjacent Cu sites. Accordingly, the C—H bond dissociation is believed to be heterolytic instead of homolytic or the Fenton type, as no ·CH₃ radicals were observed using EPR (FIG. 27). Agarwal et al., 2017; Szecsenyi et al., 2018a; Ab Rahim et al., 2013. The heterolytic dissociation of C—H bond is believed to be essential for partial oxidation of methane at high selectivities, as the other two activation mechanisms via ·CH₃ radicals are typically accompanied with over oxidation to form substantial amounts of CO₂. Compared to the case in Cu-ZSM-5, Ab Rahim et al., 2013, the Cu dimers supported on g-C₃N₄ have shorter Cu—O bond length (1.77 Å vs 1.88 Å) and smaller/Cu—O—Cu (99.6° vs) 135°, which are believed to sterically favor the heterolytic cleavage of the C—H bond and facilitate the transer of the —CH₃ group. Noticeably, the —CH₃ group can adsorb on either Cu_{α} or Cu_{β}, where the reaction bifurcates into two possible pathways. On the one hand, *CH₃ on Cu_{α} recombine with the *OOH on this site to form *CH₃OOH. On the other hand, it also can recombine with *OH on Cu_{β} to form *CH₃OH.

Desorption of these adsorbates gives rise to the corresponding methyl oxygenates. While the rate is limited by the *CH₃+*OH→*CH₃OH recombination on Cu_{β} (with a barrier of 0.72 eV), the highest barrier for the CH₃OOH pathway is found to be the desorption of *CH₃OOH (0.52 eV). Overall, the CH₃OOH pathway associated with Cu_{α} is energetically more favorable than the CH₃OH pathway with Cu_{β}, explaining the experimentally observed much higher yield of CH₃OOH than CH₃OH. The pathways as revealed in FIG. 3d emphasize the synergy among the two Cu atoms and the bridging O in catalyzing the complex reaction involving multiple molecules (e.g., CH₄+2H₂O₂→CH₃OOH+2H₂O), which is a unique feature of the carbon nitride supported dimeric copper centers. An analogous reaction mechanism also was proposed in the partial oxidation of methane with H₂O₂ catalyzed by Au—Pd colloids. Agarwal et al., 2017.

### 1.4.3 Photocatalytic Oxidation of Methane with O₂

Despite the selective oxidation of methane obtained with Cu₂@C₃N₄, the thermocatalytic reaction still relies on the use of H₂O₂ as oxidant, which is not readily available in industry. Moreover, the low CH₄ conversions (less than 1%) also limits the potential of this process for practical implementations. Considering that g-C₃N₄ is a semiconductor (with a bandgap of 2.7-2.9 eV, Wen et al., 2017; Xu and Gao, 2012) with demonstrated photocatalytic applications, Su et al., 2010, photocatalysis was evaluated to overcome the limitation of thermocatalytic reactions.

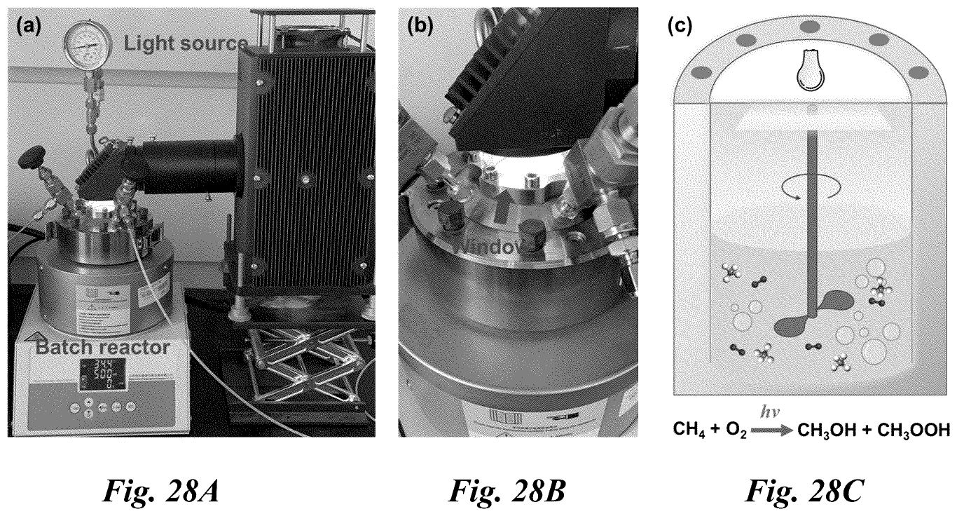

Photocatalytic oxidation of methane was carried out at 50° C. by applying near-edge excitation (300 W Xenon lamp equipped with a 420-nm bandpass filter) and using O₂ as the oxidant (FIG. 28). Without being bound to any one particular theory, it is thought that photoexcitation can efficiently activate O₂ and generate the oxygenates (*OOH and *OH), mimicking and improving the role that H₂O₂ played in the reaction. Song et al., 2019a; Song et al., 2019b; Luo et al., 2021.

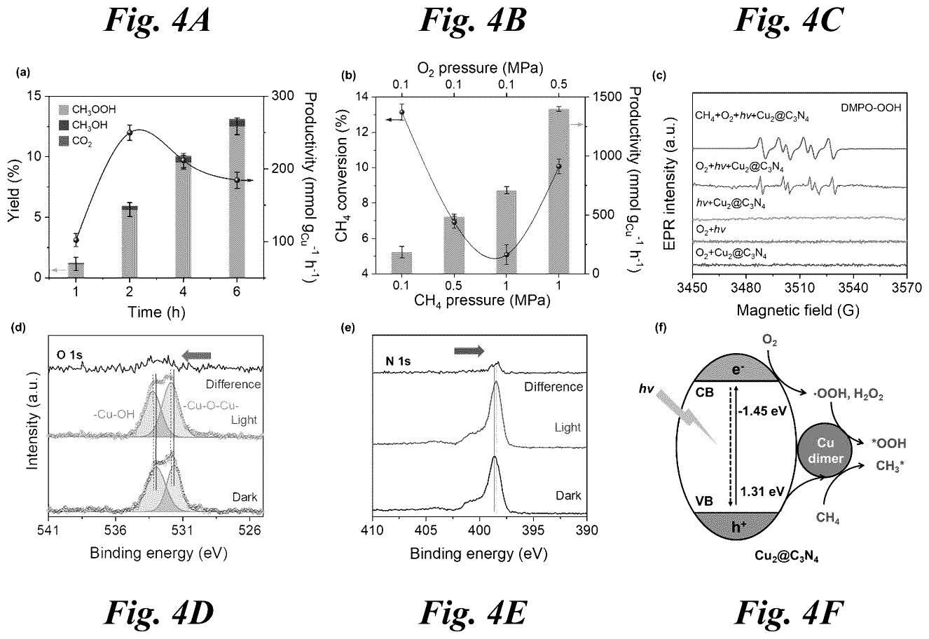

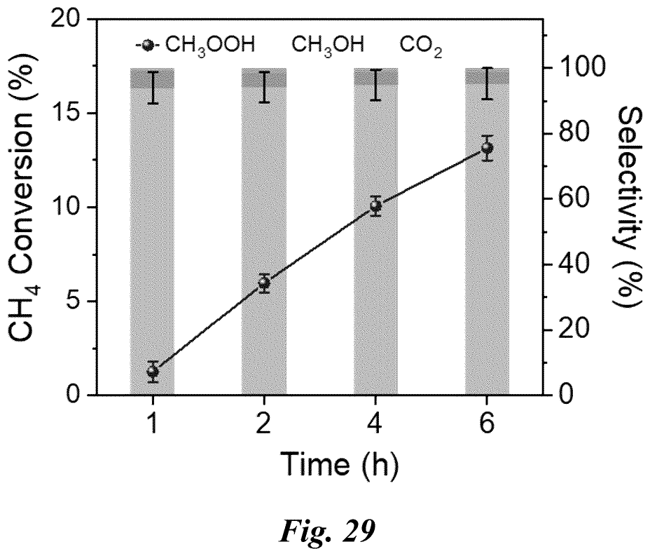

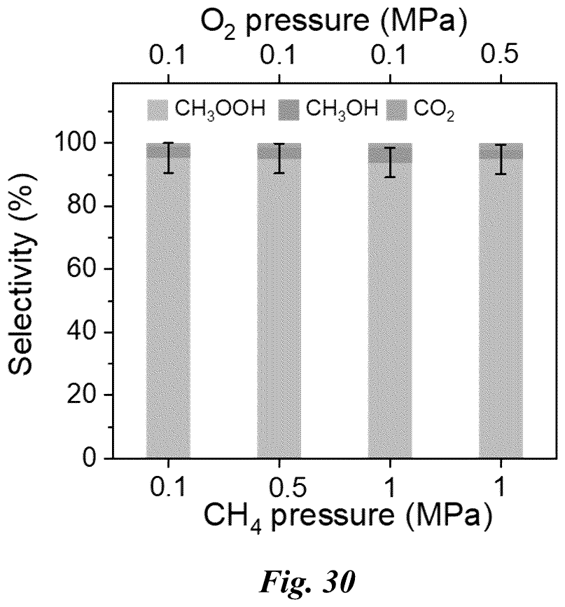

The photocatalytic reaction gave much higher conversions of methane than the thermocatalytic process, reaching 1.3% at 1 h (FIG. 4a). The methane conversion increases with time, reaching approximately 13.1% at 6 h, where the products were found to be still dominated by CH₃OOH and CH₃OH (98.9% selectivity, FIG. 29). The productivity of methyl oxygenates reached the peak value of 249.7 mmol g_{Cu}⁻¹ h⁻¹ at 2 h, representing an improvement factor of approximately 3.6 as compared to the thermocatalytic reaction. Further improvement of the productivity was obtained by raising the partial pressure of methane (P_{CH4}). As P_{CH4} increased from 0.1 to 1 MPa (at P_{O2}=0.1 MPa, while the total pressure was kept constant at 3 MPa), the productivity escalated from 184.3 to 709.8 mmol g_{Cu}⁻¹ h⁻¹, albeit with the methane conversion reducing from 13.1 to 5.1 (at 6 h, FIG. 4b and FIG. 30). The improvement of productivity at higher P_{CH4} can be ascribed to the increased concentration of dissolved methane in the aqueous solution. Jin et al., 2020. The low conversion of methane at high P_{CH4} was likely limited by the inadequacy of oxygen. At P_{O2}=0.5 MPa and P_{CH4}=1 MPa, a methane conversion of 10.1% was obtained with greater than 98% selectivity toward methyl oxygenates, corresponding to an even higher productivity of 1399.3 mmol g_{Cu}⁻¹ h⁻¹. A comprehensive comparison to the literature results under similar reaction conditions indicate that this activity represents the highest activity for partial oxidation of methane, with improvement factors of at least greater than 10 (Table 5).

TABLE 5
Comparison of photocatalytic performance for partial oxidation of CH₄ with O₂.

| | | | CH₃OOH + CH₃OH | | |
|---|---|---|---|---|---|
| | | CH₄ | | Productivity | |
| | | Conversion | Selectivity | (mmol | |
| Catalyst | Reaction conditions | (%) | (%) | gₘₑₜₐₗ⁻¹ h⁻¹) | Reference |
| Au_{0.75}/ZnO | 30° C./2 MPa/1.5 MPa CH₄/0.5 MPa O₂ | 0.05 | 99.1 | 91.4 | Zhou et al., |
| | | | | | 2020. |
| Au₁/Black P | 90° C./3.3 MPa/3 MPa CH₄/3 MPa O₂ | 0.01 | >99% | 28.4 | Luo et al., |
| | | | | | 2021. |
| 0.1% Au/ZnO | 30° C./2.1 MPa/2 MPa CH₄/0.1 MPa O₂ | 0.32 | 95.4 | 12.5 | Song et al., |
| | | | | | 2019a. |
| CuSSZ-13 | 270° C./0.1 MPa/18 kPa CH₄/0.09 kPa O₂ | 0.024 | 60 | 1.8 | Dinh, et al., |
| | | | | | 2019. |
| Bi₂WO₆ | 55° C./0.1 MPa/0.1 MPa CH₄/trace O₂ | 1.4 | 27.6 | 0.0046 | Murcia-Lopez, |
| | | | | | et al., 2014.. |
| Pd_{0.3}Cu_{0.7}O/C | 50° C./3.5 MPa/3 MPa CH₄/0.5 MPa O₂ | 0.09 | 25% | 50 | Bai, et al., |
| | | | | | 2019. |
| Au—CoOₓ/TiO₂ | 25° C./2.1 MPa/2 MPa CH₄/0.1 MPa O₂ | 0.06 | 95 | 25.4 | Song et al., |
| | | | | | 2020. |
| Cu₂@C₃N₄ | 50° C./3 MPa/0.1 MPa CH₄/0.1 MPa O₂ | 13.1 | 98.9 | 184.3 | This work |
| Cu₂@C₃N₄ | 50° C./3 MPa/1 MPa CH₄/0.5 MPa O₂ | 10.1 | 98.0 | 1399.3 | This work |

The photocatalytic oxidation of methane with O₂ was confirmed by conducting control experiments under various conditions (Table 6). In particular, the Cu₂@C₃N₄ catalyst was found to be inactive in darkness (while the other conditions were kept the same), ruling out the involvement of thermocatalytic reaction between CH₄ and O₂ in the photocatalytic studies. The photocatalytic activity of bare g-C₃N₄ also was nearly negligible, underlining the role of Cu dimers in catalyzing the related molecular transformations. The generation of active peroxide species in situ during the photocatalytic reaction was confirmed by performing EPR spectroscopic studies by also using DMPO as the radical trapping agent (FIG. 4c). The spectra recorded under visible light irradiation, in both cases with and without methane, show the fingerprints of ·OOH radicals, which can be assigned to the spin of unpaired electrons on oxygen. Hammond et al., 2012; Song et al., 2019a; Xing et al., 2021; Luo et al., 2021.

Similar to the findings from photocatalytic studies, such signals were not observed from the controls in the absence of O₂, Cu₂@C₃N₄, or light. These ·OOH radicals are likely derived from the thermal activation of H₂O₂ (as observed in the thermocatalytic studies, FIG. 25), which was produced from photocatalytic reduction of O₂ in situ. Shiraishi et al., 2014; Moon et al., 2017; Rao and Hayon, 1975. It thus becomes evident that Cu₂@C₃N₄ is not only a good thermocatalyst for partial oxidation of methane with H₂O₂, but also an exceptional photocatalyst when the oxidant is replaced by O₂.

TABLE 6
Catalytic performance of multiple control experiments for
the photocatalysis of selective CH₄ oxidation by O₂.

| Reaction conditions | Catalytic performance |
|---|---|
| CH₄ + O₂ + H₂O + hυ (no Cu₂@C₃N₄) | no activity |
| O₂ + H₂O + hυ + Cu₂@C₃N₄ (no CH₄) | no activity |
| O₂ + H₂O + hυ + C₃N₄ (no Cu dimer) | no activity |
| CH₄ + O₂ + H₂O + Cu₂@C₃N₄ (no hυ) | no activity |
| CH₄ + H₂O + Cu₂@C₅N₄hυ (no O₂) | no activity |

In addition to the reduction of O₂ to peroxides, the photon excitation also is believed to enhance the methane activation. This characteristic was revealed by using in situ irradiation X-ray photoelectron spectroscopy (ISI—XPS), Zhang et al., 2020, to examine charge transfer between the dimeric copper center and the C₃N₄ substrate (see 1.5 Methods). As shown in FIG. 4d, the XPS spectra collected on hydrated Cu₂@C₃N₄ in darkness exhibited two O 1s peaks at ca. 533.0 and 531.7 eV, which can be assigned to the oxygen binding to Cu, i.e., —Cu—OH and —Cu—O—Cu—, respectively. Akhavan et al., 2011; Bojestig et al., 2020.

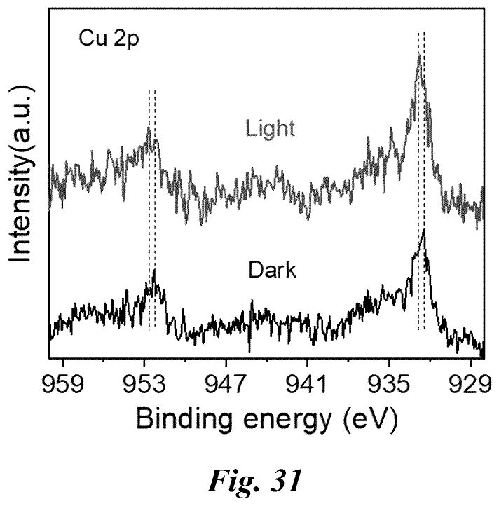

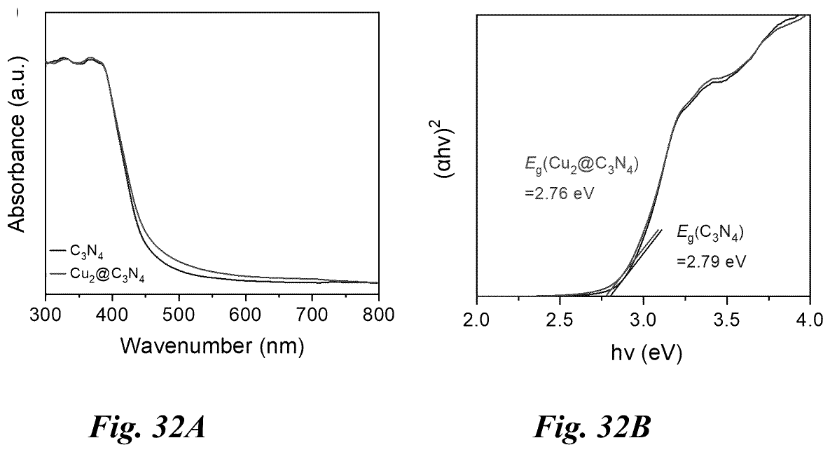

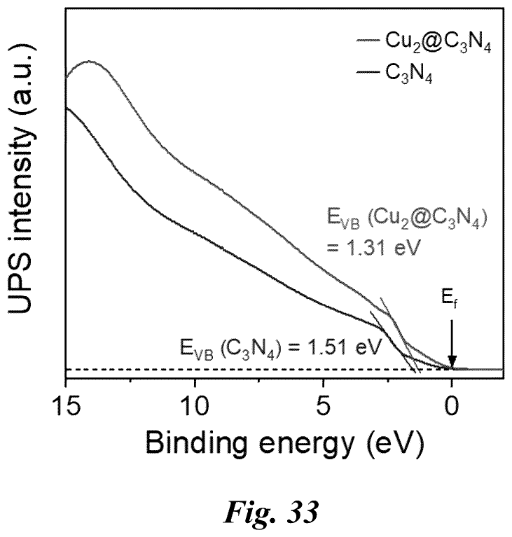

Under light irradiation (between about 400 to about 500 nm), both of these two peaks had a blue shift of approximately 0.5 eV. Similar observations were obtained at the Cu 2p edge (FIG. 31). Meanwhile, a red shift of the N Is peak associated with C₃N₄ was observed, from 398.8 eV in darkness to 398.2 eV under light irradiation (FIG. 4e). Such phenomena consistently point to the transfer of holes (rather than electrons) from the g-C₃N₄ substrate to the dicopper-oxo center, where CH₄ is activated and oxidized to form *CH₃. Meanwhile, the excited electrons in the g-C₃N₄ substrate lead to the reduction of O₂ and formation of H₂O₂, which then migrates or diffuses onto the dicopper-oxo center and gets activated to form *OOH or *OH. In the following, these oxygen species recombine with *CH₃ to form methyl oxygenates, as in the case of thermocatalytic reactions (FIG. 4f and FIG. 32-FIG. 33). Similar phenomena of charge transfer induced catalytic enhancements have previously been reported in photocatalysis using TiO₂ based photocatalysts. Dong et al., 2013; Wang et al., 2018; Xie et al., 2018.

### 1.5 Methods

### 1.5.1 Materials and Chemicals

The following chemicals were purchased and used as-received without further purification: Copper (II) chloride dihydrate (CuCl_{2·2}H₂O, ACS grade, Sigma Aldrich), 2,2,-bipyridine (C₁₀H₈N₂, reagent grade, Sigma Aldrich), oxalic acid (HO₂CCO₂H, reagent grade, Alfa Aesar), urea (NH₂CONH₂, ACS grade, Sigma Aldrich), dicyandiamide (NH₂C(═NH) NHCN, ACS grade, Sigma Aldrich), copper (II) acetylacetonate (Cu(acac)₂, ACS reagent, Sigma Aldrich), oleylamine (CH₃(CH₂)₇CH═CH(CH₂)₈NH₂, ≥98%, Sigma Aldrich), ethanol (C₂H₅OH, HPLC grade, Fisher Scientific), methanol (CH₃OH, HPLC grade, Fisher Scientific), deionized water (18.2 MΩ) was collected from an ELGA PURELAB flex apparatus.

### 1.5.2 Synthesis of Copper Dimer Complex

Solutions A, B and C were prepared by ultrasonically dispersion method, respectively. The detailed preparation process was as follows: Solution A: 1.6 mmol 272 mg CuCl₂·2H₂O was ultrasonically dispersed in 20 mL deionized water; Solution B: 1.6 mmol 248 mg 2,2,-bipyridine was ultrasonically dispersed in 10 mL methanol; Solution C: 0.8 mmol 100 mg oxalic acid was ultrasonically dispersed in 10 mL deionized water; Subsequently, adding solution B and solution C to solution A drop by drop respectively and kept stirring for 1 h. Finally, the light-blue solid was obtained by centrifugation, washing with water and methanol for three times and drying in vacuum. Reinoso et al., 2003.

### 1.5.3 Synthesis of g-C₃N₄

20 g of urea was placed to an alumina crucible (100 mL). Subsequently, the crucible was sealed with multiple layers of tin foil and put into a muffle furnace with the heating program from 50° C. to 550° C. for 2 h at the rate of 20° C. min-1. The obtained powder was further subjected to the above calcination operation, with the difference being that the heating rate was kept at 5° C. min-1 and the retention time at 550° C. was 3 h. Finally, the yellowish-white powder was obtained. 1.5.4 Synthesis of Cu₂@C₃N₄

Solution A: 0.5 g g-C₃N₄ was ultrasonically dispersed in 50 mL methanol solution; Solution B: 42 mg copper dimer was ultrasonically dispersed in 5 mL methanol solution; Solution B was added dropwise added to solution A and was stirred at room temperature for 24 h, and the obtained solid was calcined in muffle furnace with the heating program from 50° C. to 250° C. for 10 h at the rate of 2° C. min-1. Finally, the blue-yellow solid was obtained.

### 1.5.5 Synthesis of Cu₁@g-C₃N₄ (Wu et al., 2019)

3 g dicyandiamide and 340 mg CuCl₂·2H₂O were grounded to be-well mixed, then spread in an alumina crucible (100 mL) with a cap covered. The crucibles were places in a muffle furnace, and gradually heated to 550° C. for 8 hours with the ramping rate of 5° C. min-1 and then cooled down.

### 1.5.6 Material Characterization

X-Ray Diffraction (XRD) patterns were obtained from a PANalytical X'Pert³ X-ray diffractometer equipped with a Cu Kα radiation source (2=1.5406 Å). Nitrogen adsorption measurements were measured on a Micromeritics ASAP 2010 instrument with the samples degassed under vacuum at 300° C. for 4 h. Specific surface area (SSA) was calculated using the Brunauer-Emmett-Teller (BET) theory. The Cu contents were determined by inductively coupled plasma mass spectrometry (ICP-MS) using a PerkinElmer Elan DRC II Quadrupole ICP-MS after dissolution of the samples in aqua regia. High angle annular dark field (HAADF) STEM images were acquired using a JEOL TEM/STEM ARM 200CF (equipped with an Oxford X-max 100TLE windowless X-ray detector) at a 22-mrad probe convergence angle and a 90-mrad inner-detector angle. The analysis of surface elements was performed on X-ray photoelectron spectroscopy (XPS), Thermo Fisher Scientific Escalab 250Xi spectrometer with Al Kα radiation as the excitation source. Fourier Transform Infrared Spectroscopy were carried out on ThermoNicolet Nexus 670. Diffuse reflectance ultraviolet-visible (UV-Vis) spectra were collected on a Shimadzu UV-2450 spectrometer equipped with an integrating sphere attachment using BaSO₄ as the reference. FTIR Spectrometer Ultraviolet photoelectron spectroscopy (UPS) measurements were performed on an ESCALAB 250 UPS instrument with a He Iα gas discharge lamp operating at 21.22 eV and a total instrumental energy resolution of 90-120 meV.

XAS experiments were performed at the 10-BM beamline at the Advanced Photon Source (APS) at Argonne National Laboratory. Samples were pressed into a stainless-steel sample holder. All measurements were performed at the Cu K edge (8.9789 keV) in transmission mode in fast scan from 250 eV below the edge to 800 eV above the edge. Spectra processing, including background removal and normalization were performed on ATHENA module in Demeter package. The extraction of structural parameters and fitting of the DFT optimized models of fresh and spent Cu₂@C₃N₄ samples were performed on ARTEMIS module. For the optimized structure, EXAFS data were fit from k=2.7 to 10 Å-1 (dk=2) and R=1−3.2 Å with a Hanning window.

Electron Paramagnetic Resonance (EPR) measurements were performed on a Bruker EMX EPR spectrometer at X-band frequency (9.46 GHz). 5,5′-Dimethyl-1-pyrroline-N-oxide (DMPO) was used as the spin-trapping agent, which can capture the radicals ·CH₃, ·OOH and ·OH. As for the detection of ·OOH and ·OH, methanol and DI H₂O were used respectively, due to the DMPO-OOH is not stable in H₂O, would be quickly converted to DMPO-OH.

The in-situ irradiation X-Ray photoelectron spectroscopy (ISI—XPS) was carried out on AXIS SUPRA (Kratos Analytical Inc, Shimadazu) coupled with a continuous tunable wavelength light optical fiber (PLS-EM 150, Beijing Perfectlight Co. Ltd.). The wavelength of irradiation light was set at 400-500 nm to mimicking the visible light. The measurement setup is developed to monitor the photoelectron transfer process. Before measurement, the hydrated Cu₁@g-C₃N₄ was obtained by pretreatment of fresh Cu₁@g-C₃N₄ by water.

### 1.5.7 Thermocatalytic Measurements

The selective methane oxidation was performed in a high-pressure Parr reactor. 0.2 mmol H₂O₂ dissolved in 10 mL deionized H₂O was used as the oxidizing agent. 50 mg of catalyst powder was added to the aqueous solution. After evacuating the air left in reactor by flowing methane (0.1 MPa) and purging for five times, the system then was pressurized with argon to 3 MPa. The solution was vigorously stirred at 1500 rpm, meanwhile heated to 50° C. Both temperature and pressure were well controlled and kept constant during catalysis. The reaction time of all experiments was strictly controlled at certain time (e.g., 30 mins, 1 or 2 h) after the temperature of solution reaches a pre-set temperature. After the reaction, the reactor was set in an ice bath to cool down immediately, the solution was kept being stirred at 1500 rpm.

Upon completely cooling the reaction down to ice bath temperature, the gas components (i.e., CH₄, CO₂) were injected and determined with gas chromatograph equipped with a BID detector (GC-2010 plus, Shimadzu). Before analysis, the gas in the autoclave was used to sweep the GC lines for 20 s. The solution consisting of liquid products was filtered from catalyst powder. The liquid products, including CH₃OOH, CH₃OH and others, were quantitatively analyzed with 1H-NMR. Typically, 0.7 mL of sample and 0.1 mL of D₂O were placed in an NMR tube along with 4,4-dimethyl-4-silapentane-1-sulfonic acid (DSS, 8-0 ppm) as the internal standard. During NMR measurements, a solvent suppression program was run to minimize the signal originating from H₂O. A typical 1H-NMR spectrum is provided in FIG. 12. The identified oxygenated products were methanol (δ=3.34 ppm) and methyl hydroperoxide (δ=3.85 ppm). Ratios of peak areas of methanol or methyl peroxide to peak area of DSS were calculated. The products are determined by using the standard curves of methanol and methyl hydroperoxide as provided in FIG. 13, FIG. 14.

The H₂O₂ concentration was measured by a traditional cerium sulfate Ce(SO₄)₂ titration method based on the mechanism that a yellow solution of Ce⁴⁺ would be reduced by H₂O₂ to colorless Ce³⁺ (2Ce⁴⁺+H₂O₂→2Ce³⁺+2H⁺+O₂). Thus, the concentration of Ce⁴⁺ before and after the reaction can be measured by ultraviolet-visible spectroscopy. The wavelength used for the measurement was 316 nm. The standard curve of H₂O₂ is provided in FIG. 21.

### 1.5.8 Photocatalytic Studies

The photocatalytic methane oxidation reaction tests were conducted in a 50-mL batch-reactor equipped with a quartz window to allow light irradiation. Typically, 50-mg catalyst was dispersed in 10-mL deionized water by ultrasonication for 10 min. Then the mixture was added into the reaction cell, and the reaction cell was placed in the batch-reactor. The batch-reactor was purged with 0.1-MPa CH₄ and 0.1-MPa O₂ for five times to exhaust air, then the reactor was pressurized with argon to 3 MPa. To study the influence of different CH₄ or O₂ partial pressure on the photocatalytic reaction, 0.5- or 1-MPa CH₄ with 0.1-MPa O₂ or 0.1-MPa CH₄ with 0.5 MPa O₂ also were applied. Subsequently, the reactor was stirred at 50° C. under the light irradiation provided by a 300-W xenon lamp (MC-XS500, Testmart), equipped with a 420-nm optical filter (Ceaulight), the light intensity was controlled at 100 mW/cm. A thermocouple was inserted into the solution to directly detect the temperature of the liquid solution. During the process, the temperature was maintained at 50° C. After the reaction, the reactor was cooled in an ice bath to a temperature below 10° C. The analysis of products followed the same protocol as shown above. The conversion of CH₄, the selectivity of products, and the mass reaction rate were calculated according to the following equations:

$${mol}_{CH 4 , input} = \frac{{mol}_{Ar}}{GC Peak area of Ar} \times f ( correction factor ) \times GC Peak of {CH}_{4} \\ Conversion = \frac{mol ( {CH}_{3} OOH ) + mol ( {CH}_{3} OH ) + mol ( {CO}_{2} )}{mol ( {CH}_{4 , input} )} \times 100 \% \\ Selectivity = \frac{mol ( specific product )}{mol ( {CH}_{3} OOH ) + mol ( {CH}_{3} OH ) + mol ( {CO}_{2} )} \times 100 \% \\ Productivity ( mmol {g}^{- 1} {h}^{- 1} ) = \frac{mol ( specific product )}{weight of Cu ( g ) \times reaction time ( h )} \times 100 \%$$

### 1.5.9 Computational Methods

In this work, all the simulations were carried out for the direct synthesis of CH₃OOH and CH₃OH within the framework of the spin-polarized generalized gradient approximation with the Perdew-Burke-Ernzerh, Perdew, et al., 1996, of functional in the VASP code. Kresse and Furthmuller, 1996. The cutoff energy of plane-wave basis expansion was set to 400 eV. The approach of project-augmented-wave (PAW), Kresse and Joubert, 1999, was exploited to describe the interaction between core-electron and valence electron. The Methfessel-Paxton-approach with a fermi smearing width of 0.1 eV was used to determine partial occupancies on electronic states. Electronic convergence was set to 10-5 eV, and geometries were converged to less than 0.05 eV/Å. All the possible surfaces were constructed with 2×2×1 Monkhorst-Pack k-point mesh sampling which is well tested. Chow and Vosko, 1980. The effect of vdW interaction is significant for the reaction mechanism in our precious study. Yao et al., 2019; Wei et al., 2019. Therefore, the DFT-D3 method of Grimme et al., 2011, was utilized to calculate all the energetics and structures of the intermediates and transition states. All the surfaces were relaxed with a 15 Å vacuum region. The transition states (TSs) were searched using the method called a constrained optimization scheme. Liu and Hu, 2003; Zhang et al., 1999. The TSs were confirmed by two rules: (i) all forces on atoms vanish; (ii) the total energy is a maximum along the reaction coordinate but a minimum with respect to the rest of the degrees of freedom. Vibrational frequency analyses were performed to confirm the integrity of TSs. Cortright and Dumesic, 2001.

### 1.6 Summary

The presently disclosed subject matter provides novel dimeric copper catalysts for the partial oxidation of methane. These catalysts were synthesized by immobilization of a copper-dimer organometallic complex on graphitic carbon nitride. Dicopper-oxo centers were characterized as anchoring on this substrate via Cu—N bonding. The derived Cu₂@C₃N₄ catalysts were first examined for thermocatalytic oxidation of methane with H₂O₂, and then studied for photocatalytic reactions with O₂ being used as the oxidant. Enhanced catalytic activities were demonstrated in both cases as compared to the other reported catalysts under similar reaction conditions, achieving improvement factors of more than an order of magnitude. Synergy of the bridging oxygen, the two copper sites and the semiconducting C₃N₄ substrate has been revealed to promote H₂O₂ and O₂ activation and the heterolytic scission of CH₄. The presently disclosed subject matter highlights the great potential of carbon nitride supported dimeric copper centers in catalyzing redox chemical reactions.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Nisbet, E. G. D., E. J. Bousquet, P. Methane on the Rise-Again. Science 343, 493-495 (2014).
Choudhary, T. V., Choudhary, V. R. Energy-efficient syngas production through, catalytic oxy-methane reforming reactions. Angew. Chem. Int. Edit. 47, 1828-1847 (2008).
Tang, P. Z., Q. J. Wu, Z. X. Ma, D. Methane activation: the past and future. Energ. Environ. Sci. 7, 2580-2591 (2014).
Buzcu-Guven, B. & Harriss, R. Extent, impacts and remedies of global gas flaring and venting. Carbon. Manag. 3, 95-108 (2012).
Julian-Duran, L. M., Ortiz-Espinoza, A. P., El-Halwagi, M. M. & Jimenez-Gutierrez, A. Techno-Economic Assessment and Environmental Impact of Shale Gas Alternatives to Methanol. ACS Sustain. Chem. Eng. 2, 2338-2344 (2014).
Farrell, B. L. I., V. O. Linic, S. A Viewpoint on Direct Methane Conversion to Ethane and Ethylene Using Oxidative Coupling on Solid Catalysts. ACS Catal. 6, 4340-4346 (2016).
Hammond, C. F., M. M. Ab Rahim, M. H. Thetford, A. He, Q. Jenkins, R. L. Dimitratos, N. Lopez-Sanchez, J. A. Dummer, N. F. Murphy, D. M. Carley, A. F. Taylor, S. H. Willock, D. J. Stangland, E. E. Kang, J. Hagen, H. Kiely, C. J. Hutchings, G. J. Direct Catalytic Conversion of Methane to Methanol in an Aqueous Medium by using Copper-Promoted Fe-ZSM-5. Angew. Chem. Int. Edit. 51, 5129-5133 (2012).
Woertink, J. S. S., P. J. Groothaert, M. H. Vance, M. A. Sels, B. F. Schoonheydt, R. A. Solomon, E. I. A [Cu₂O]²⁺ core in Cu-ZSM-5, the active site in the oxidation of methane to methanol. PNAS 106, 18908-18913 (2009).
Pappas, D. K. M., A. Dyballa, M. Kvande, K. Teketel, S. Lomachenko, K. A. Baran, R. Glatzel, P. Arstad, B. Berlier, G. Lamberti, C. Bordiga, S. Olsbye, U. Svelle, S. Beato, P. Borfecchia, E. The Nuclearity of the Active Site for Methane to Methanol Conversion in Cu-Mordenite: A Quantitative Assessment. J. Am. Chem. Soc. 140, 15270-15278 (2018).
Groothaert, M. H. S., P. J. Sels, B. F. Jacobs, P. A. Schoonheydt, R. A. Selective oxidation of methane by the bis(m-oxo)dicopper core stabilized on ZSM-5 and mordenite zeolites. J. Am. Chem. Soc. 127, 1394-1395 (2005).
Narsimhan, K. I., K. Dinh, K. Roman-Leshkov, Y. Catalytic Oxidation of Methane into Methanol over Copper-Exchanged Zeolites with Oxygen at Low Temperature. ACS Central Sci. 2, 424-429 (2016).
Dinh, K. T. S., M. M. Narsimhan, K. Serna, P. Meyer, R. J. Dinca, M. Roman-Leshkov, Y. Continuous Partial Oxidation of Methane to Methanol Catalyzed by Diffusion-Paired Copper Dimers in Copper-Exchanged Zeolites. J. Am. Chem. Soc. 141, 11641-11650 (2019).
Koishybay, A. S., Shantz, D. F. Water Is the Oxygen Source for Methanol Produced in Partial Oxidation of Methane in a Flow Reactor over Cu—SSZ-13. J. Am. Chem. Soc. 142, 11962-11966 (2020).
Palkovits, R., Antonietti, M., Kuhn, P., Thomas, A. & Schuth, F. Solid Catalysts for the Selective Low-Temperature Oxidation of Methane to Methanol. Angew. Chem. Int. Edit. 48, 6909-6912 (2009).
Jones, C. J. et al. Selective oxidation of methane to methanol catalyzed, with C—H activation, by homogeneous, cationic gold. Angew. Chem. Int. Edit. 43, 4626-4629 (2004).
Shan, J. J. L., M. W. Allard, L. F. Lee, S. S. Flytzani-Stephanopoulos, M. Mild oxidation of methane to methanol or acetic acid on supported isolated rhodium catalysts. Nature 551, 605-608 (2017).
Sushkevich, V. L. P., D. Ranocchiari, M. van Bokhoven, J. A. Selective anaerobic oxidation of methane enables direct synthesis of methanol. Science 356, 523-527 (2017).
Grundner, S. M., M. A. C. Li, G. Tromp, M. Pidko, E. A. Hensen, E. J. M. Jentys, A. Sanchez-Sanchez, M. Lercher, J. A. Single-site trinuclear copper oxygen clusters in mordenite for selective conversion of methane to methanol. Nat. Commun. 6 (2015).
Agarwal, N. F., S. J. Mc Vicker, R. U. Althahban, S. M. Dimitratos, N. He, Q. Morgan, D. J. Jenkins, R. L. Willock, D. J. Taylor, S. H. Kiely, C. J. Hutchings, G. J. Aqueous Au—Pd colloids catalyze selective CH₄ oxidation to CH₃OH with O₂ under mild conditions. Science 358, 223-226 (2017).
Jin, Z. W., L. Zuidema, E. Mondal, K. Zhang, M. Zhang, J. Wang, C. T. Meng, X. J. Yang, H. Q. Mesters, C. Xiao, F. S. Hydrophobic zeolite modification for in situ peroxide formation in methane oxidation to methanol. Science 367, 193-197 (2020).
Song, H. et al. Direct and Selective Photocatalytic Oxidation of CH₄ to Oxygenates with O₂ on Cocatalysts/ZnO at Room Temperature in Water. J. Am. Chem. Soc. 141, 20507-20515 (2019).
Song, H., Meng, X. G., Wang, Z. J., Liu, H. M. & Ye, J. H. Solar-Energy-Mediated Methane Conversion. Joule 3, 1606-1636 (2019).
Song, H. et al. Selective Photo-oxidation of Methane to Methanol with Oxygen over Dual-Cocatalyst-Modified Titanium Dioxide. ACS Catal. 10, 14318-14326 (2020).
Szecsenyi, A., Li, G. N., Gascon, J. & Pidko, E. A. Mechanistic Complexity of Methane Oxidation with H₂O₂ by Single-Site Fe/ZSM-5 Catalyst. ACS Catal. 8, 7961-7972 (2018).
Wen, J. Q. X., J. Chen, X. B. Li, X. A review on g-C₃N₄-based photocatalysts. Appl. Surf. Sci. 391, 72-123 (2017).
Xu, Y. & Gao, S. P. Band gap of C₃N₄ in the GW approximation. Int. J. Hydrogen Energ. 37, 11072-11080 (2012).
Su, F. Z. et al. mpg-C₃N₄-Catalyzed Selective Oxidation of Alcohols Using O₂ and Visible Light. J. Am. Chem. Soc. 132, 16299-16301 (2010).
Shi, Z. S., Yang, W. Q., Gu, Y. T., Liao, T. & Sun, Z. Q. Metal-Nitrogen-Doped Carbon Materials as Highly Efficient Catalysts: Progress and Rational Design. Adv. Sci. 7 (2020).
Reinoso, S. V., P. Lezama, L. Luque, A. Gutierrez-Zorrilla, J. M. A novel organic-inorganic hybrid based on a dinuclear copper complex supported on a keggin polyoxometalate. Inorg. Chem. 42, 3709-3711 (2003).
Martin, D. J. et al. Highly Efficient Photocatalytic H₂ Evolution from Water using Visible Light and Structure-Controlled Graphitic Carbon Nitride. Angew. Chem. Int. Edit. 53, 9240-9245 (2014).
Zhao, Y. Y. et al. Stable iridium dinuclear heterogeneous catalysts supported on metal-oxide substrate for solar water oxidation. PNAS 115, 2902-2907 (2018).
Gerasimova, T. P. & Katsyuba, S. A. Bipyridine and phenanthroline IR-spectral bands as indicators of metal spin state in hexacoordinated complexes of Fe(II), Ni(II) and Co(II). Dalton. T. 42, 1787-1797 (2013).
Mane, G. P. T., S. N. Lakhi, K. S. Ilbeygi, H. Ravon, U. Al-Bahily, K. Mori, T. Park, D. H. Vinu, A. Highly Ordered Nitrogen-Rich Mesoporous Carbon Nitrides and Their Superior Performance for Sensing and Photocatalytic Hydrogen Generation. Angew Chem. Int. Edit. 56, 8481-8485 (2017).
Chen, Z. P. M., S. Vorobyeva, E. Leary, R. K. Hauert, R. Furnival, T. Ramasse, Q. M. Thomas, J. M. Midgley, P. A. Dontsova, D. Antonietti, M. Pogodin, S. Lopez, N. Perez-Ramirez, J. Stabilization of Single Metal Atoms on Graphitic Carbon Nitride. Adv. Funct. Mater. 27 (2017).
Fox, E. B. V., S. Engelhard, M. H. Chin, Y. H. Miller, J. T. Kropf, J. Song, C. S. Characterization of CeO₂-supported Cu—Pd bimetallic catalyst for the oxygen-assisted water-gas shift reaction. J. Catal. 260, 358-370 (2008).
Xie, P. F. et al. Bridging adsorption analytics and catalytic kinetics for metal-exchanged zeolites. Nat. Catal. 4, 144-156 (2021).
Tsai, M. L. et al. [Cu₂O]²⁺ Active Site Formation in Cu-ZSM-5: Geometric and Electronic Structure Requirements for N₂O Activation. J. Am. Chem. Soc. 136, 3522-3529 (2014).
Ab Rahim, M. H. F., M. M. Jenkins, R. L. Hammond, C. He, Q. Dimitratos, N. Lopez-Sanchez, J. A. Carley, A. F. Taylor, S. H. Willock, D. J. Murphy, D. M. Kiely, C. J. Hutchings, G. J. Oxidation of Methane to Methanol with Hydrogen Peroxide Using Supported Gold-Palladium Alloy Nanoparticles. Angew. Chem. Int. Edit. 52, 1280-1284 (2013).
Kulkarni, A. R., Zhao, Z. J., Siahrostami, S., Norskov, J. K. & Studt, F. Cation-exchanged zeolites for the selective oxidation of methane to methanol. Catal. Sci. Technol. 8, 114-123 (2018).
Yashnik, S. A., Boltenkov, V. V., Babushkin, D. E., Taran, O. P. & Parmon, V. N. Methane Oxidation by H₂O₂ over Different Cu-Species of Cu-ZSM-5 Catalysts. Top. Catal. 63, 203-221 (2020).
Zheng, J. Y., J. Y. Ortuno, M. A. Fulton, J. L. Gutierrez, O. Y. Camaioni, D. M. Motkuri, R. K. Li, Z. Y. Webber, T. E. Mehdi, B. L. Browning, N. D. Penn, R. L. Farha, O. K. Hupp, J. T. Truhlar, D. G. Cramer, C. J. Lercher, J. A. Selective Methane Oxidation to Methanol on Cu-Oxo Dimers Stabilized by Zirconia Nodes of an NU-1000 Metal-Organic Framework. J. Am. Chem. Soc. 141, 9292-9304 (2019).
Qu, Y. T. et al. Direct transformation of bulk copper into copper single sites via emitting and trapping of atoms. Nat. Catal. 1, 781-786 (2018).
Ravi, M. et al. Misconceptions and challenges in methane-to-methanol over transition-metal-exchanged zeolites. Nat. Catal. 2, 485-494 (2019).
Lu, Z. Y. et al. High-efficiency oxygen reduction to hydrogen peroxide catalysed by oxidized carbon materials. Nat. Catal. 1, 156-162 (2018).
Xing, Y. C. et al. Fe/Fe₃C Boosts H₂O₂ Utilization for Methane Conversion Overwhelming O₂ Generation. Angew Chem. Int. Edit. 60, 2-9 (2021).
Szecsenyi, A., Li, G. N., Gascon, J. & Pidko, E. A. Unraveling reaction networks behind the catalytic oxidation of methane with H₂O₂ over a mixed-metal MIL-53 (Al,Fe) MOF catalyst. Chem. Sci. 9 (2018).
Osadchii, D. Y. O.—S., A. I. Szecsenyi, A. Li, G. N. Nasalevich, M. A. Dugulan, J. A. Crespo, P. S. Hensen, E. J. M. Veber, S. L. Fedin, M. V. Sankar, G. Pidko, E. A. Gascon, J. Isolated Fe Sites in Metal Organic Frameworks Catalyze the Direct Conversion of Methane to Methanol. ACS Catal. 8, 5542-5548 (2018).
Schneider, J. T., Firak, D. S., Ribeiro, R. R. & Peralta-Zamora, P. Use of scavenger agents in heterogeneous photocatalysis: truths, half-truths, and misinterpretations. Phys. Chem. Chem. Phys. 22, 15723-15733 (2020).
Hori, Y., Shiota, Y., Tsuji, T., Kodera, M. & Yoshizawa, K. Catalytic Performance of a Dicopper-Oxo Complex for Methane Hydroxylation. Inorg. Chem. 57, 8-11 (2018).
Zhou, P. et al. Strengthening reactive metal-support interaction to stabilize high-density Pt single atoms on electron-deficient g-C₃N₄ for boosting photocatalytic H₂ production. Nano Energy 56, 127-137 (2019).
Xiao, X. D. et al. A Promoted Charge Separation/Transfer System from Cu Single Atoms and C₃N₄ Layers for Efficient Photocatalysis. Adv. Mater. 32 (2020).
Guo, Y. Q. et al. A strategy for enhancing the photoactivity of g-C₃N₄ based single-atom catalysts via sulphur doping: a theoretical study. Phys. Chem. Chem. Phys. 23, 6632-6640 (2021).
Tong, T., He, B. W., Zhu, B. C., Cheng, B. & Zhang, L. Y. First-principle investigation on charge carrier transfer in transition-metal single atoms loaded g-C₃N₄. Appl. Surf. Sci. 459, 385-392 (2018).
Luo, L. H. et al. Water enables mild oxidation of methane to methanol on gold single-atom catalysts. Nat. Commun. 12, 1218 (2021).
Shiraishi, Y. et al. Highly Selective Production of Hydrogen Peroxide on Graphitic Carbon Nitride (g-C₃N₄) Photocatalyst Activated by Visible Light. ACS Catal. 4, 774-780 (2014).
Moon, G. H. et al. Eco-Friendly Photochemical Production of H₂O₂ through O₂ Reduction over Carbon Nitride Frameworks Incorporated with Multiple Heteroelements. ACS Catal. 7, 2886-2895 (2017).
Rao, P. S. & Hayon, E. Redox Potentials of Free-Radicals. IV. Superoxide and Hydroperoxy Radicals Single Bond ·O₂⁻ and Single Bond ·HO₂. J. Phys. Chem. 79, 397-402 (1975).
Zhang, P. et al. Photogenerated Electron Transfer Process in Heterojunctions: In Situ Irradiation XPS. Small Methods 4 (2020).
Akhavan, O., Azimirad, R., Safa, S. & Hasani, E. CuO/Cu(OH)₂ hierarchical nanostructures as bactericidal photocatalysts. J. Mater. Chem. 21, 9634-9640 (2011).
Bojestig, E., Cao, Y. & Nyborg, L. Surface chemical analysis of copper powder used in additive manufacturing. Surf. Interface Anal. 52, 1104-1110 (2020).
Dong, F. et al. In Situ Construction of g-C₃N₄/g-C₃N₄ Metal-Free Heterojunction for Enhanced Visible-Light Photocatalysis. ACS Appl. Mater. Inter. 5, 11392-11401 (2013).
Wang, D., Sheng, T., Chen, J. F., Wang, H. F. & Hu, P. Identifying the key obstacle in photocatalytic oxygen evolution on rutile TiO₂. Nat. Catal. 1, 291-299 (2018).
Xie, J. J. et al. Highly selective oxidation of methane to methanol at ambient conditions by titanium dioxide-supported iron species. Nat. Catal. 1, 889-896 (2018).
S. V. Reinoso, P. Lezama, L. Luque, A. Gutierrez-Zorrilla, J. M., A novel organic-inorganic hybrid based on a dinuclear copper complex supported on a keggin polyoxometalate. Inorg. Chem. 42, 3709-3711 (2003).
Q. Wu et al., Cu@g-C₃N₄: An Efficient Single-Atom Electrocatalyst for NO Electrochemical Reduction with Suppressed Hydrogen Evolution. J. Phys. Chem. C 123, 31043-31049 (2019).
J. P. Perdew, K. Burke, M. Ernzerhof, Generalized gradient approximation made simple. Phys. Rev. Lett. 77, 3865-3868 (1996).
G. Kresse, J. Furthmuller, Efficiency of ab-initio total energy calculations for metals and semiconductors using a plane-wave basis set. Comp. Mater. Sci. 6, 15-50 (1996).
G. Kresse, D. Joubert, From ultrasoft pseudopotentials to the projector augmented-wave method. Phys. Rev. B 59, 1758-1775 (1999).
H. C. Chow, S. H. Vosko, Special Points for Two-Dimensional Brillouin-Zone or Wigner-Seitz Cell Integrations. Can. J. Phys. 58, 497-503 (1980).
Z. H. Yao, C. X. Guo, Y. Mao, P. Hu, Quantitative Determination of C—C Coupling Mechanisms and Detailed Analyses on the Activity and Selectivity for Fischer-Tropsch Synthesis on Co (0001): Microkinetic Modeling with Coverage Effects. ACS Catal. 9, 5957-5973 (2019).
Z. Z. Wei et al., Optimizing Alkyne Hydrogenation Performance of Pd on Carbon in Situ Decorated with Oxygen-Deficient TiO₂ by Integrating the Reaction and Diffusion. ACS Catal. 9, 10656-10667 (2019).
S. Grimme, S. Ehrlich, L. Goerigk, Effect of the Damping Function in Dispersion Corrected Density Functional Theory. J. Comput. Chem. 32, 1456-1465 (2011).
Z. P. Liu, P. Hu, General rules for predicting where a catalytic reaction should occur on metal surfaces: A density functional theory study of C—H and C—O bond breaking/making on flat, stepped, and kinked metal surfaces. J. Am. Chem. Soc. 125, 1958-1967 (2003).
C. J. Zhang, P. J. Hu, A. Alavi, A general mechanism for CO oxidation on close-packed transition metal surfaces. J. Am. Chem. Soc. 121, 7931-7932 (1999).
R. D. Cortright, J. A. Dumesic, Kinetics of heterogeneous catalytic reactions: Analysis of reaction schemes. Adv. Catal. 46, 161-264 (2001).
W. C. Zhou et al., Highly selective aerobic oxidation of methane to methanol over gold decorated zinc oxide via photocatalysis. J. Mater. Chem. A 8, 13277-13284 (2020).
L. H. Luo et al., Water enables mild oxidation of methane to methanol on gold single-atom catalysts. Nat. Commun. 12, 1218 (2021).
S. Murcia-Lopez, K. Villa, T. Andreu, J. R. Morante, Partial Oxidation of Methane to Methanol Using Bismuth-Based Photocatalysts. ACS Catal. 4, 3013-3019 (2014).
S. X. Bai, Y. Xu, P. T. Wang, Q. Shao, X. Q. Huang, Activating and Converting CH₄ to CH₃OH via the CuPdO₂/CuO Nanointerface. ACS Catal. 9, 6938-6944 (2019).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A catalyst composite comprising a copper dimer having two copper atoms bridged by an oxygen atom, wherein each copper atom is coordinated to two nitrogen atoms of a carbon nitride substrate.

2. The catalyst composite of claim 1, wherein the carbon nitride substrate comprises one or more of graphitic carbon nitride, α-carbon nitride, β-carbon nitride, cubic carbon nitride, pseudocubic carbon nitride, and combinations thereof.

3.-4. (canceled)

5. The catalyst composite of claim 1, comprising a loading of Cu of about 0.25 wt % to about 0.5 wt % of the total weight of the composite.

6. The catalyst composite of claim 1, wherein the composite has an N 1s X-ray photoelectron spectroscopy (XPS) spectrum exhibiting a binding energy ranging from about 397 eV to about 408 eV comprising four peaks centered at about 398.6 eV, about 399.4 eV, about 401.0 eV, and about 404.5 eV.

7. The catalyst composite of claim 1, wherein the composite has a Cu 2p X-ray photoelectron spectroscopy (XPS) spectrum exhibiting peaks at 932.5 eV and 952.3 eV.

8. The catalyst composite of claim 1, wherein the composite has an Absorption Near Edge Spectroscopy (XANES) Cu K-edge spectrum exhibiting a pre-edge transition at about 8,984 eV.

9. The catalyst composite of claim 1, wherein the two copper atoms have an oxidation state between +1 and +2.

10. (canceled)

11. The catalyst composite of claim 1, wherein an average distance between the two copper atoms is between about 0.18 nm to about 0.38 nm.

12. (canceled)

13. A method for preparing the catalyst composite of claim 1, the method comprising:
(a) providing a copper-dimer organometallic precursor;
(b) mixing the copper dimer organometallic precursor with a carbon nitride substrate to form a mixture; and
(c) heating the mixture to form a catalyst composite comprising a copper dimer having two copper atoms bridged by an oxygen atom, wherein each copper atom is coordinated to two nitrogen atoms of a carbon nitride substrate.

14. The method of claim 13, wherein the copper-dimer organometallic precursor comprises an (oxalato) (bipyridine) copper (II) complex (Cu₂(bpy)₂(μ-ox)]Cl₂).

15.-18. (canceled)

19. The method of claim 13, comprising heating the mixture from about 50° C. to about 250° C. for about 10 hours at a rate of about 2° C. min⁻¹.

20. A method for oxidizing methane, the method comprising contacting methane with a catalyst composite of claim 1 in the presence of an oxidizing agent.

21. The method of claim 20, wherein the oxidizing agent comprises H₂O₂

22. The method of claim 21, wherein the method comprises thermocatalytic oxidation of CH₄.

23. The method of claim 22, wherein the methane, catalyst composite, and oxidizing agent are maintained at a predetermined pressure and temperature for a period of time.

24. The method of claim 20, wherein the oxidizing agent comprises O₂.

25. The method of claim 24, wherein the method comprises photocatalytic oxidation of CH₄.

26. The method of claim 25, wherein the methane, catalyst composite, and oxidizing agent are irradiated with visible light at a predetermined pressure and temperature for a period of time.

27. The method of claim 20, comprising forming one or more methyl oxygenates.

28. The method of claim 27, wherein the one or more methyl oxygenates are selected from methanol (CH₃OH) and methyl hydroperoxide (CH₃OOH).

29. (canceled)
